(19) United States
(12) Patent Application Publication
(10) Pub. No.: US 2010/0268000 A1
(43) Pub. Date: Oct. 21, 2010

(54) Compositions and Methods for Fermentation of Biomass

(75) Inventors: Sarad Parekh, Grafton, MA (US); William G. LaTouf, Ashland, MA (US)

Correspondence Address: WILSON, SONSINI, GOODRICH & ROSATI, 650 PAGE MILL ROAD, PALO ALTO, CA, 94304-1050, US

(73) Assignee: QTEROS, INC., Marlborough, MA (US)

(21) Appl. No.: 12/763,966

(22) Filed: Apr. 20, 2010

Related U.S. Application Data
(60) Provisional application No. 61/171,077, filed on Apr. 20, 2009.
(60) Provisional application No. 61/171,831, filed on Apr. 22, 2009.
(60) Provisional application No. 61/221,519, filed on Jun. 29, 2009.

(51) Int. Cl. C07C 31/08; C12P 7/10; C12M 1/00

(52) U.S. Cl. 568/840; 435/165; 435/289.1

(57) ABSTRACT

In one aspect, this invention relates to production of useful fermentation end-products from biomass through simultaneous hydrolysis and fermentation by a microorganism, such as Clostridium phytofermentans. The invention also relates to the development of a process for efficient pretreatment and conversion of lignocellulosic biomass to end-products with high conversion efficiency (yield). In another aspect, methods for producing a fermentation end-product by fermenting hexose (C6) and pentose (C5) sugars with a microorganism, such as Clostridium phytofermentans are disclosed herein.

### CROSS-REFERENCE TO RELATED APPLICATIONS

This application claims priority to U.S. Provisional Application Ser. Nos. 61/171,077, filed Apr. 20, 2009; 61/171,831, filed Apr. 22, 2009; and 61/221,519 filed on Jun. 29, 2009, which are herein incorporated by reference in their entirety.

### BACKGROUND OF THE INVENTION

This invention relates to production of useful fermentation end-products from biomass through simultaneous hydrolysis and fermentation of sugars and oligomers. The invention also relates to the development of a process for efficient pretreatment and conversion of lignocellulosic biomass to end-products with high conversion efficiency (yield).

Various forms of biomass have potential as renewable feedstocks for ethanol production due to their enormous availability and low cost. However, there still exists an unmet need for a low cost, robust method of utilizing available feedstock for the generation of fermentation end-products such as ethanol and other chemical compounds or biofuels.

Fermentation of biomass to produce biofuels such as alcohols (e.g. methanol, ethanol, butanol, or propanol) can provide much needed solutions for the world energy problem. Lignocellulosic biomass has cellulose and hemicellulose as two major components. Hydrolysis of these components results in both hexose (C6) as well as pentose (C5) sugars. Biomass conversion efficiency is highly dependent on the range of carbohydrates that can be utilized by the organism used in the biomass to fuel conversion process. In particular, an inability to utilize both hexose (e.g. cellobiose, glucose) and pentose (e.g. arabinose, xylose) sugars for conversion into ethanol can dramatically limit the total amount of biofuel or other chemicals that can be generated from a given quantity of biomass. Therefore, to obtain a high conversion efficiency of lignocellulosic biomass to ethanol (yield), it is important to be able to successfully ferment both hexose and pentose sugars into ethanol.

However, fermentation of pentose sugars (xylose and arabinose) is still a technological bottleneck for ethanol production from biomass. This limitation can lead to ethanol production at low efficiencies, low maximum achievable biofuel titer in a fermentation reaction, and low biofuel productivity. Further, much of the carbohydrate content of biomass can be lost through the solubilization of pentose sugars during pretreatment. Generally, lower yields and low productivity result in higher production costs, which can translate into competitive disadvantages which may not be offset by other characteristics of the microorganism.

### SUMMARY OF THE INVENTION

In one aspect the invention discloses a method for producing one or more fermentation end-products by fermenting a lignocellulosic biomass comprising hexose and pentose saccharides with a first microorganism, wherein said first microorganism simultaneously hydrolyses and ferments the lignocellulosic biomass to produce a fermentation end-product. In one embodiment at least one of the fermentation end-products is ethanol and wherein the ethanol is produced to a titer of at least about 45 g/L. In another embodiment the first microorganism is a Clostridium strain. In another embodiment the Clostridium strain is Clostridium phytofermentans. In another embodiment the method further comprises the fermentation of hexose and pentose saccharides using a second microorganism. In another embodiment the second microorganism is Saccharomyces cerevisiae, C. thermocellum, C. acetobutylicum, C. cellovorans, or Zymomonas mobilis. In another embodiment the hexose saccharides comprise carbohydrates selected from the group consisting of cellulose, hemicellulose, starch, mannan, fructose, glucose, galactose, rhamnose, and mannose. In another embodiment the pentose saccharides comprise carbohydrates selected from the group consisting of xylan, hemicellulose, xylose, and arabinose. In another embodiment the Clostridium phytofermentans is nonrecombinant or recombinant In another embodiment the Clostridium phytofermentans comprises one or more heterologous polynucleotides. In another embodiment one or more medium supplements comprising hexose or pentose saccharides is added to the medium during the growth of the first microorganism. In another embodiment the hexose or pentose saccharides are added in relation to the amount of sugar converted by the first microorganism to other compounds. In another embodiment the method comprises pretreatment of the biomass. In another embodiment the pretreatment comprises steam explosion or hot water extraction, exposure to acid or alkaline conditions. In another embodiment the method comprises adding a fermentation medium supplement, wherein said fermentation medium supplement is fatty acid, a surfactant, a chelating agent, vitamins, minerals, pH modifiers, yeast extract, and salts. In another embodiment the first microorganism simultaneously ferments said hexose and pentose saccharides. In another embodiment the method comprises adding one or more enzymes, wherein the one or more enzymes are not derived from first microorganism. In another embodiment the one or more enzymes is a cellulase, a hemicellulase, a galacturonase, a pectate lyase, a carbohydrase, a xylanase, a glucanase, and endoglucanase, an exoglucanase, a glucosidase, an amylase, a phytase, or a laccase. In another embodiment the hexose and pentose saccharides comprise malt syrup, corn steep liquor, distillers dried grains or corn steep solids. In another embodiment the method further comprises fed-batch fermentation of biomass with bolus addition of biomass solids. In another embodiment the biomass solids are recovered using a sieve. In another embodiment the sieve comprises a plurality of apertures between about 150-250 microns in diameter.

In another aspect the invention discloses a biofuel product produced by culturing a strain of Clostridium phytofermentans in a medium comprising a lignocellulosic biomass; wherein the Clostridium phytofermentans simultaneously hydrolyses and ferments the lignocellulosic biomass.

In another aspect the invention discloses a method of producing ethanol, the method comprising the steps of: culturing a strain of Clostridium phytofermentans in a medium comprising a lignocellulosic biomass; wherein the Clostridium phytofermentans simultaneously hydrolyses and ferments the lignocellulosic biomass; producing ethanol at a yield greater than about 45 g/L. In one embodiment the method further comprises adding one or more medium supplements to the medium during the growth of the Clostridium phytofermentans, wherein one or more of the medium supplements comprises one or more hexose and/or pentose sugar compounds, and the one or more sugar compounds are added in relation to the amount of sugar converted by the Clostridium phytofermentans to other compounds.

In another aspect the invention discloses a system for the production of a fermentation end product comprising: a fermentation vessel; a lignocellulosic biomass; and a first microorganism that simultaneously hydrolyses and ferments the lignocellulosic biomass; wherein the fermentation vessel is adapted to provide suitable conditions for the simultaneous hydrolysis and fermentation of the lignocellulosic biomass. In one embodiment further comprises a medium supplement comprising with hexose and pentose saccharides. In another embodiment the first microorganism is a Clostridium strain. In another embodiment the Clostridium strain is Clostridium phytofermentans. In another embodiment the Clostridium phytofermentans comprises one or more heterologous polynucleotides. In another embodiment the biomass is pretreated by steam explosion or hot water extraction, exposure to acid or alkaline conditions before contact with the first microorganism. In another embodiment the pre-treated biomass is further treated with one or more enzymes not derived from the first microorganism. In another embodiment the hexose and pentose saccharides comprise one or more of corn steep solids, corn steep liquor, malt syrup, xylan, cellulose, hemicellulose, fructose, glucose, mannose, rhamnose, or xylose. In another embodiment the fermentation medium further comprises one or more enzymes not derived from the first microorganism. In another embodiment the fermentation medium further comprises a fermentation medium supplement selected from the group consisting of a fatty acid, a surfactant, a chelating agent, vitamins, minerals, pH modifiers, yeast extract, and salts. In another embodiment the system further comprises a second microorganism. In another embodiment the second microorganism is Saccharomyces cerevisiae, C. thermocellum, C. acetobutylicum, C. cellovorans, or Zymomonas mobilis.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the cumulative total quantity of treated biomass solids fed into a fermentation reaction over a period of about 15 days.

FIG. 2 is a pie chart depicting the carbohydrate composition of corn stover biomass treated by size reduction and alkali according to methods described herein.

FIGS. 3A-3B depict sugar and ethanol concentration profiles for a fermentation reaction at the indicated time points. FIG. 3A depicts the sugar and ethanol concentration profile for the simultaneous fermentation of cellobiose and xylose. FIG. 3B depicts the sugar and ethanol concentration profile for the simultaneous fermentation of glucose and xylose.

FIG. 4 depicts normalized uptake and utilization of the indicated hexose (glucose, cellobiose) and pentose (xylose) sugars in a fermentation reaction at the indicated time points.

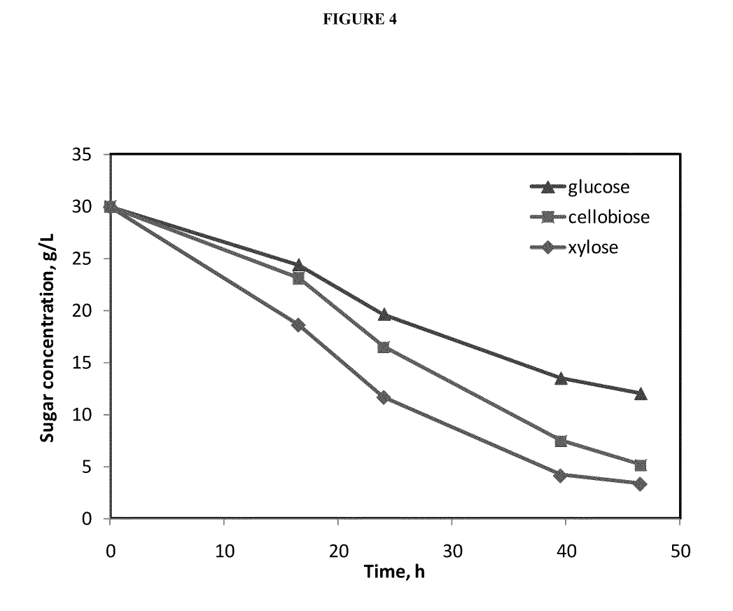

FIG. 5 depicts sugar and ethanol concentration profile for the simultaneous fermentation of xylose, cellobiose, and starch to ethanol at the indicated time points.

FIG. 6 depicts sugar and ethanol concentration profile for the simultaneous fermentation of glucose, xylose, and arabinose into ethanol at the indicated time points.

FIG. 7 depicts ethanol concentration profile for the simultaneous fermentation of starch, cellulose, xylan, and cellobiose.

FIG. 8 depicts a method for producing fermentation end products from biomass by first treating biomass with an acid at elevated temperature and pressure in a hydrolysis unit.

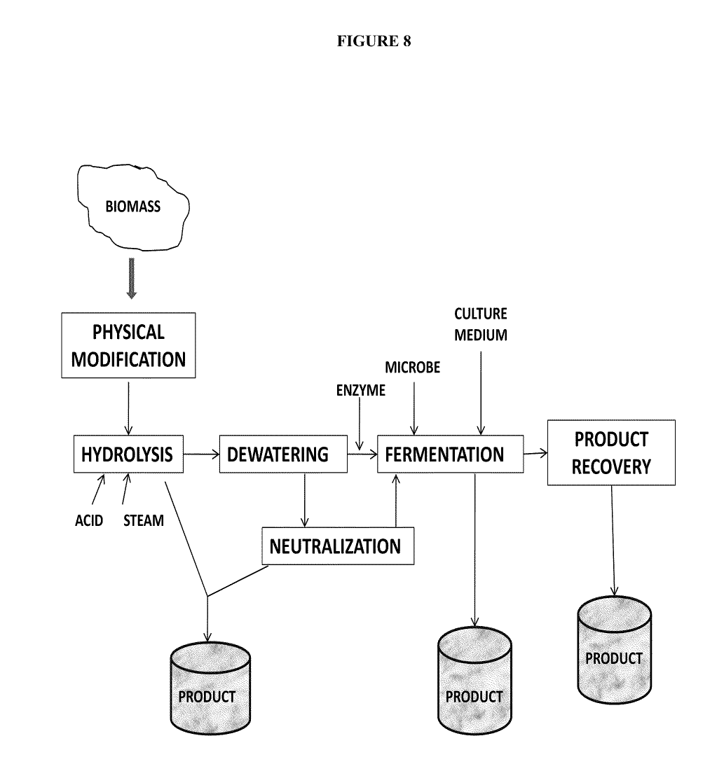

FIG. 9 depicts a method for producing fermentation end products from biomass by charging biomass to a fermentation vessel.

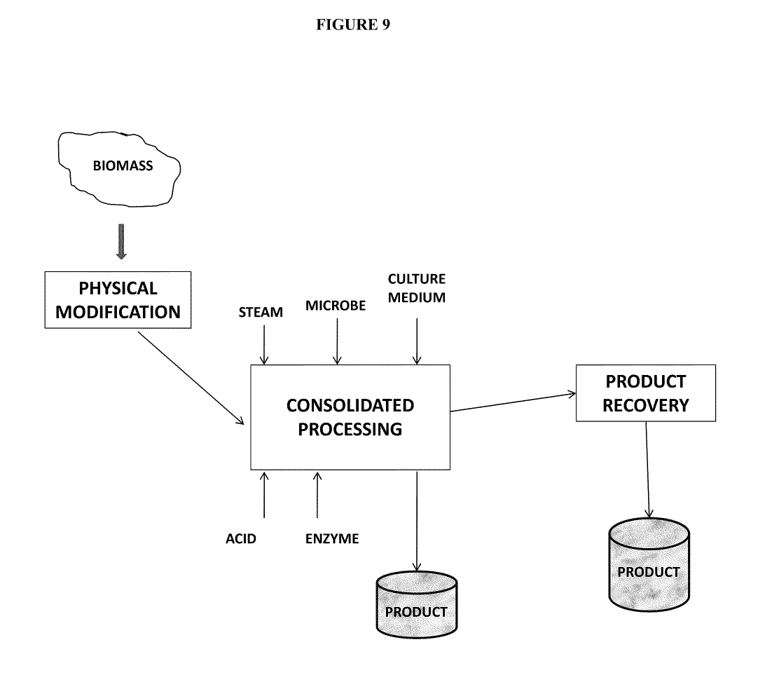

FIG. 10 discloses pretreatments that produce hexose or pentose saccharides or oligomers that are then unprocessed or processed further and either, fermented separately or together.

FIG. 11 is a map of the plasmid pIMPT1029 used to transform Clostridium phytofermentans.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The following description and examples illustrate embodiments of the present invention in detail. Those of skill in the art will recognize that there are numerous variations and modifications of this invention that are encompassed within its scope. Accordingly, the description of a preferred embodiment should not be deemed to limit the scope of the present invention

### DEFINITIONS

Unless characterized differently, technical and scientific terms used herein have the same meaning as commonly understood by one of ordinary skill in the art to which this invention belongs.

The term “about” in relation to a reference numerical value includes a range of values plus or minus 15% from that value. For example the amount “about 10” includes amounts from 8.5 to 11.5.

The term “fuel” or “biofuel” as used herein has its ordinary meaning as known to those skilled in the art and can include one or more compounds suitable as liquid fuels, gaseous fuels, reagents, chemical feedstocks and includes, but is not limited to hydrocarbons, hydrogen, methane, biodiesel, hydroxy compounds such as alcohols (e.g. ethanol, butanol, propanol, methanol, etc.), and carbonyl compounds such as aldehydes and ketones (e.g. acetone, formaldehyde, 1-propanal, etc.).

The term “fermentation end-product” or “end-product” as used herein has its ordinary meaning as known to those skilled in the art and can include one or more biofuels, chemical additives, processing aids, food additives, organic acids (e.g. acetic, lactic, formic, citric acid etc.), derivatives of organic acids such as esters (e.g. wax esters, glycerides, etc.) and other functional compounds including, but not limited to, 1,2-propanediol, 1,3-propanediol, lactic acid, formic acid, acetic acid, succinic acid, pyruvic acid, enzymes such as cellulases, polysaccharases, lipases, proteases, ligninases, and hemicellulases and can be present as a pure compound, a mixture, or an impure or diluted form. Further examples of fermentation end-products include, but are not limited to, 1,4 diacids (succinic, fumaric and malic), 2,5 furan dicarboxylic acid, 3 hydroxy propionic acid, aspartic acid, glucaric acid, glutamic acid, itaconic acid, levulinic acid, 3-hydroxybutyrolactone, glycerol, sorbitol, xylitol/arabinitol, butanediol, butanol, methane, methanol, ethane, ethene, ethanol, n-propane, 1-propene, 1-propanol, propanal, acetone, propionate, n-butane, 1-butene, 1-butanol, butanal, butanoate, isobutanal, isobutanol, 2-methylbutanal, 2-methylbutanol, 3-methylbutanal, 3-methylbutanol, 2-butene, 2-butanol, 2-butanone, 2,3-butanediol, 3-hydroxy-2-butanone, 2,3-butanedione, ethylbenzene, ethenylbenzene, 2-phenylethanol, phenylacetaldehyde, 1-phenylbutane, 4-phenyl-1-butene, 4-phenyl-2-butene, 1-phenyl-2-butene, 1-phenyl-2-butanol, 4-phenyl-2-butanol, 1-phenyl-2-butanone, 4-phenyl-2-butanone, 1-phenyl-2,3-butandiol, 1-phenyl-3-hydroxy-2-butanone, 4-phenyl-3-hydroxy-2-butanone, 1-phenyl-2,3-butanedione, n-pentane, ethylphenol, ethenylphenol, 2-(4-hydroxyphenyl)ethanol, 4-hydroxyphenylacetaldehyde, 1-(4-hydroxyphenyl) butane, 4-(4-hydroxyphenyl)-1-butene, 4-(4-hydroxyphenyl)-2-butene, 1-(4-hydroxyphenyl)-1-butene, 1-(4-hydroxyphenyl)-2-butanol, 4-(4-hydroxyphenyl)-2-butanol, 1-(4-hydroxyphenyl)-2-butanone, 4-(4-hydroxyphenyl)-2-butanone, 1-(4-hydroxyphenyl)-2,3-butandiol, 1-(4-hydroxyphenyl)-3-hydroxy-2-butanone, 4-(4-hydroxyphenyl)-3-hydroxy-2-butanone, 1-(4-hydroxyphenyl)-2,3-butanonedione, indolylethane, indolylethene, 2-(indole-3-)ethanol, n-pentane, 1-pentene, 1-pentanol, pentanal, pentanoate, 2-pentene, 2-pentanol, 3-pentanol, 2-pentanone, 3-pentanone, 4-methylpentanal, 4-methylpentanol, 2,3-pentanediol, 2-hydroxy-3-pentanone, 3-hydroxy-2-pentanone, 2,3-pentanedione, 2-methylpentane, 4-methyl-1-pentene, 4-methyl-2-pentene, 4-methyl-3-pentene, 4-methyl-2-pentanol, 2-methyl-3-pentanol, 4-methyl-2-pentanone, 2-methyl-3-pentanone, 4-methyl-2,3-pentanediol, 4-methyl-2-hydroxy-3-pentanone, 4-methyl-3-hydroxy-2-pentanone, 4-methyl-2,3-pentanedione, 1-phenylpentane, 1-phenyl-1-pentene, 1-phenyl-2-pentene, 1-phenyl-3-pentene, 1-phenyl-2-pentanol, 1-phenyl-3-pentanol, 1-phenyl-2-pentanone, 1-phenyl-3-pentanone, 1-phenyl-2,3-pentanediol, 1-phenyl-2-hydroxy-3-pentanone, 1-phenyl-3-hydroxy-2-pentanone, 1-phenyl-2,3-pentanedione, 4-methyl-1-phenylpentane, 4-methyl-1-phenyl-1-pentene, 4-methyl-1-phenyl-2-pentene, 4-methyl-1-phenyl-3-pentene, 4-methyl-1-phenyl-3-pentanol, 4-methyl-1-phenyl-2-pentanol, 4-methyl-1-phenyl-3-pentanone, 4-methyl-1-phenyl-2-pentanone, 4-methyl-1-phenyl-2,3-pentanediol, 4-methyl-1-phenyl-2,3-pentanedione, 4-methyl-1-phenyl-3-hydroxy-2-pentanone, 4-methyl-1-phenyl-2-hydroxy-3-pentanone, 1-(4-hydroxyphenyl) pentane, 1-(4-hydroxyphenyl)-1-pentene, 1-(4-hydroxyphenyl)-2-pentene, 1-(4-hydroxyphenyl)-3-pentene, 1-(4-hydroxyphenyl)-2-pentanol, 1-(4-hydroxyphenyl)-3-pentanol, 1-(4-hydroxyphenyl)-2-pentanone, 1-(4-hydroxyphenyl)-3-pentanone, 1-(4-hydroxyphenyl)-2,3-pentanediol, 1-(4-hydroxyphenyl)-2-hydroxy-3-pentanone, 1-(4-hydroxyphenyl)-3-hydroxy-2-pentanone, 1-(4-hydroxyphenyl)-2,3-pentanedione, 4-methyl-1-(4-hydroxyphenyl) pentane, 4-methyl-1-(4-hydroxyphenyl)-2-pentene, 4-methyl-1-(4-hydroxyphenyl)-3-pentene, 4-methyl-1-(4-hydroxyphenyl)-1-pentene, 4-methyl-1-(4-hydroxyphenyl)-3-pentanol, 4-methyl-1-(4-hydroxyphenyl)-2-pentanol, 4-methyl-1-(4-hydroxyphenyl)-3-pentanone, 4-methyl-1-(4-hydroxyphenyl)-2-pentanone, 4-methyl-1-(4-hydroxyphenyl)-2,3-pentanediol, 4-methyl-1-(4-hydroxyphenyl)-2,3-pentanedione, 4-methyl-1-(4-hydroxyphenyl)-3-hydroxy-2-pentanone, 4-methyl-1-(4-hydroxyphenyl)-2-hydroxy-3-pentanone, 1-indole-3-pentane, 1-(indole-3)-1-pentene, 1-(indole-3)-2-pentene, 1-(indole-3)-3-pentene, 1-(indole-3)-2-pentanol, 1-(indole-3)-3-pentanol, 1-(indole-3)-2-pentanone, 1-(indole-3)-3-pentanone, 1-(indole-3)-2,3-pentanediol, 1-(indole-3)-2-hydroxy-3-pentanone, 1-(indole-3)-3-hydroxy-2-pentanone, 1-(indole-3)-2,3-pentanedione, 4-methyl-1-(indole-3-)pentane, 4-methyl-1-(indole-3)-2-pentene, 4-methyl-1-(indole-3)-3-pentene, 4-methyl-1-(indole-3)-1-pentene, 4-methyl-2-(indole-3)-3-pentanol, 4-methyl-1-(indole-3)-2-pentanol, 4-methyl-1-(indole-3)-3-pentanone, 4-methyl-1-(indole-3)-2-pentanone, 4-methyl-1-(indole-3)-2,3-pentanediol, 4-methyl-1-(indole-3)-2,3-pentanedione, 4-methyl-1-(indole-3)-3-hydroxy-2-pentanone, 4-methyl-1-(indole-3)-2-hydroxy-3-pentanone, n-hexane, 1-hexene, 1-hexanol, hexanal, hexanoate, 2-hexene, 3-hexene, 2-hexanol, 3-hexanol, 2-hexanone, 3-hexanone, 2,3-hexanediol, 2,3-hexanedione, 3,4-hexanediol, 3,4-hexanedione, 2-hydroxy-3-hexanone, 3-hydroxy-2-hexanone, 3-hydroxy-4-hexanone, 4-hydroxy-3-hexanone, 2-methylhexane, 3-methylhexane, 2-methyl-2-hexene, 2-methyl-3-hexene, 5-methyl-1-hexene, 5-methyl-2-hexene, 4-methyl-1-hexene, 4-methyl-2-hexene, 3-methyl-3-hexene, 3-methyl-2-hexene, 3-methyl-1-hexene, 2-methyl-3-hexanol, 5-methyl-2-hexanol, 5-methyl-3-hexanol, 2-methyl-3-hexanone, 5-methyl-2-hexanone, 5-methyl-3-hexanone, 2-methyl-3,4-hexanediol, 2-methyl-3,4-hexanedione, 5-methyl-2,3-hexanediol, 5-methyl-2,3-hexanedione, 4-methyl-2,3-hexanediol, 4-methyl-2,3-hexanedione, 2-methyl-3-hydroxy-4-hexanone, 2-methyl-4-hydroxy-3-hexanone, 5-methyl-2-hydroxy-3-hexanone, 5-methyl-3-hydroxy-2-hexanone, 4-methyl-2-hydroxy-3-hexanone, 4-methyl-3-hydroxy-2-hexanone, 2,5-dimethylhexane, 2,5-dimethyl-2-hexene, 2,5-dimethyl-3-hexene, 2,5-dimethyl-3-hexanol, 2,5-dimethyl-3-hexanone, 2,5-dimethyl-3,4-hexanediol, 2,5-dimethyl-3,4-hexanedione, 2,5-dimethyl-3-hydroxy-4-hexanone, 5-methyl-1-phenylhexane, 4-methyl-1-phenylhexane, 5-methyl-1-phenyl-1-hexene, 5-methyl-1-phenyl-2-hexene, 5-methyl-1-phenyl-3-hexene, 4-methyl-1-phenyl-1-hexene, 4-methyl-1-phenyl-2-hexene, 4-methyl-1-phenyl-3-hexene, 5-methyl-1-phenyl-2-hexanol, 5-methyl-1-phenyl-3-hexanol, 4-methyl-1-phenyl-2-hexanol, 4-methyl-1-phenyl-3-hexanol, 5-methyl-1-phenyl-2-hexanone, 5-methyl-1-phenyl-3-hexanone, 4-methyl-1-phenyl-2-hexanone, 4-methyl-1-phenyl-3-hexanone, 5-methyl-1-phenyl-2,3-hexanediol, 4-methyl-1-phenyl-2,3-hexanediol, 5-methyl-1-phenyl-3-hydroxy-2-hexanone, 5-methyl-1-phenyl-2-hydroxy-3-hexanone, 4-methyl-1-phenyl-3-hydroxy-2-hexanone, 4-methyl-1-phenyl-2-hydroxy-3-hexanone, 5-methyl-1-phenyl-2,3-hexanedione, 4-methyl-1-phenyl-2,3-hexanedione, 4-methyl-1-(4-hydroxyphenyl)hexane, 5-methyl-1-(4-hydroxyphenyl)-1-hexene, 5-methyl-1-(4-hydroxyphenyl)-2-hexene, 5-methyl-1-(4-hydroxyphenyl)-3-hexene, 4-methyl-1-(4-hydroxyphenyl)-1-hexene, 4-methyl-1-(4-hydroxyphenyl)-2-hexene, 4-methyl-1-(4-hydroxyphenyl)-3-hexene, 5-methyl-1-(4-hydroxyphenyl)-2-hexanol, 5-methyl-1-(4-hydroxyphenyl)-3-hexanol, 4-methyl-1-(4-hydroxyphenyl)-2-hexanol, 4-methyl-1-(4-hydroxyphenyl)-3-hexanol, 5-methyl-1-(4-hydroxyphenyl)-2-hexanone, 5-methyl-1-(4-hydroxyphenyl)-3-hexanone, 4-methyl-1-(4-hydroxyphenyl)-2-hexanone, 4-methyl-1-(4-hydroxyphenyl)-3-hexanone, 5-methyl-1-(4-hydroxyphenyl)-2,3-hexanediol, 4-methyl-1-(4-hydroxyphenyl)-2,3-hexanediol, 5-methyl-1-(4-hydroxyphenyl)-3-hydroxy-2-hexanone, 5-methyl-1-(4-hydroxyphenyl)-2-hydroxy-3-hexanone, 4-methyl-1-(4-hydroxyphenyl)-3-hydroxy-2-hexanone, 4-methyl-1-(4-hydroxyphenyl)-2-hydroxy-3-hexanone, 5-methyl-1-(4-hydroxyphenyl)-2,3-hexanedione, 4-methyl-1-(4-hydroxyphenyl)-2,3-hexanedione, 4-methyl-1-(indole-3-)hexane, 5-methyl-1-(indole-3)-1-hexene, 5-methyl-1-(indole-3)-2-hexene, 5-methyl-1-(indole-3)-3-hexene, 4-methyl-1-(indole-3)-1-hexene, 4-methyl-1-(indole-3)-2-hexene, 4-methyl-1-(indole-3)-3-hexene, 5-methyl-1-(indole-3)-2-hexanol, 5-methyl-1-(indole-3)-3-hexanol, 4-methyl-1-(indole-3)-2-hexanol, 4-methyl-1-(indole-3)-3-hexanol, 5-methyl-1-(indole-3)-2-hexanone, 5-methyl-1-(indole-3)-3-hexanone, 4-methyl-1-(indole-3)-2-hexanone, 4-methyl-1-(indole-3)-3-hexanone, 5-methyl-1-(indole-3)-2,3-hexanediol, 4-methyl-1-(indole-3)-2,3-hexanediol, 5-methyl-1-(indole-3)-3-hydroxy-2-hexanone, 5-methyl-1-(indole-3)-2-hydroxy-3-hexanone, 4-methyl-1-(indole-3)-3-hydroxy-2-hexanone, 4-methyl-1-(indole-3)-2-hydroxy-3-hexanone, 5-methyl-1-(indole-3)-2,3-hexanedione, 4-methyl-1-(indole-3)-2,3-hexanedione, n-heptane, 1-heptene, 1-heptanol, heptanal, heptanoate, 2-heptene, 3-heptene, 2-heptanol, 3-heptanol, 4-heptanol, 2-heptanone, 3-heptanone, 4-heptanone, 2,3-heptanediol, 2,3-heptanedione, 3,4-heptanediol, 3,4-heptanedione, 2-hydroxy-3-heptanone, 3-hydroxy-2-heptanone, 3-hydroxy-4-heptanone, 4-hydroxy-3-heptanone, 2-methylheptane, 3-methylheptane, 6-methyl-2-heptene, 6-methyl-3-heptene, 2-methyl-3-heptene, 2-methyl-2-heptene, 5-methyl-2-heptene, 5-methyl-3-heptene, 3-methyl-3-heptene, 2-methyl-3-heptanol, 2-methyl-4-heptanol, 6-methyl-3-heptanol, 5-methyl-3-heptanol, 3-methyl-4-heptanol, 2-methyl-3-heptanone, 2-methyl-4-heptanone, 6-methyl-3-heptanone, 5-methyl-3-heptanone, 3-methyl-4-heptanone, 2-methyl-3,4-heptanediol, 2-methyl-3,4-heptanedione, 6-methyl-3,4-heptanediol, 6-methyl-3,4-heptanedione, 5-methyl-3,4-heptanediol, 5-methyl-3,4-heptanedione, 2-methyl-3-hydroxy-4-heptanone, 2-methyl-4-hydroxy-3-heptanone, 6-methyl-3-hydroxy-4-heptanone, 6-methyl-4-hydroxy-3-heptanone, 5-methyl-3-hydroxy-4-heptanone, 5-methyl-4-hydroxy-3-heptanone, 2,6-dimethylheptane, 2,5-dimethylheptane, 2,6-dimethyl-2-heptene, 2,6-dimethyl-3-heptene, 2,5-dimethyl-2-heptene, 2,5-dimethyl-3-heptene, 3,6-dimethyl-3-heptene, 2,6-dimethyl-3-heptanol, 2,6-dimethyl-4-heptanol, 2,5-dimethyl-3-heptanol, 2,5-dimethyl-4-heptanol, 2,6-dimethyl-3,4-heptanediol, 2,6-dimethyl-3,4-heptanedione, 2,5-dimethyl-3,4-heptanediol, 2,5-dimethyl-3,4-heptanedione, 2,6-dimethyl-3-hydroxy-4-heptanone, 2,6-dimethyl-4-hydroxy-3-heptanone, 2,5-dimethyl-3-hydroxy-4-heptanone, 2,5-dimethyl-4-hydroxy-3-heptanone, n-octane, 1-octene, 2-octene, 1-octanol, octanal, octanoate, 3-octene, 4-octene, 4-octanol, 4-octanone, 4,5-octanediol, 4,5-octanedione, 4-hydroxy-5-octanone, 2-methyloctane, 2-methyl-3-octene, 2-methyl-4-octene, 7-methyl-3-octene, 3-methyl-3-octene, 3-methyl-4-octene, 6-methyl-3-octene, 2-methyl-4-octanol, 7-methyl-4-octanol, 3-methyl-4-octanol, 6-methyl-4-octanol, 2-methyl-4-octanone, 7-methyl-4-octanone, 3-methyl-4-octanone, 6-methyl-4-octanone, 2-methyl-4,5-octanediol, 2-methyl-4,5-octanedione, 3-methyl-4,5-octanediol, 3-methyl-4,5-octanedione, 2-methyl-4-hydroxy-5-octanone, 2-methyl-5-hydroxy-4-octanone, 3-methyl-4-hydroxy-5-octanone, 3-methyl-5-hydroxy-4-octanone, 2,7-dimethyloctane, 2,7-dimethyl-3-octene, 2,7-dimethyl-4-octene, 2,7-dimethyl-4-octanol, 2,7-dimethyl-4-octanone, 2,7-dimethyl-4,5-octanediol, 2,7-dimethyl-4,5-octanedione, 2,7-dimethyl-4-hydroxy-5-octanone, 2,6-dimethyloctane, 2,6-dimethyl-3-octene, 2,6-dimethyl-4-octene, 3,7-dimethyl-3-octene, 2,6-dimethyl-4-octanol, 3,7-dimethyl-4-octanol, 2,6-dimethyl-4-octanone, 3,7-dimethyl-4-octanone, 2,6-dimethyl-4,5-octanediol, 2,6-dimethyl-4,5-octanedione, 2,6-dimethyl-4-hydroxy-5-octanone, 2,6-dimethyl-5-hydroxy-4-octanone, 3,6-dimethyloctane, 3,6-dimethyl-3-octene, 3,6-dimethyl-4-octene, 3,6-dimethyl-4-octanol, 3,6-dimethyl-4-octanone, 3,6-dimethyl-4,5-octanediol, 3,6-dimethyl-4,5-octanedione, 3,6-dimethyl-4-hydroxy-5-octanone, n-nonane, 1-nonene, 1-nonanol, nonanal, nonanoate, 2-methylnonane, 2-methyl-4-nonene, 2-methyl-5-nonene, 8-methyl-4-nonene, 2-methyl-5-nonanol, 8-methyl-4-nonanol, 2-methyl-5-nonanone, 8-methyl-4-nonanone, 8-methyl-4,5-nonanediol, 8-methyl-4,5-nonanedione, 8-methyl-4-hydroxy-5-nonanone, 8-methyl-5-hydroxy-4-nonanone, 2,8-dimethylnonane, 2,8-dimethyl-3-nonene, 2,8-dimethyl-4-nonene, 2,8-dimethyl-5-nonene, 2,8-dimethyl-4-nonanol, 2,8-dimethyl-5-nonanol, 2,8-dimethyl-4-nonanone, 2,8-dimethyl-5-nonanone, 2,8-dimethyl-4,5-nonanediol, 2,8-dimethyl-4,5-nonanedione, 2,8-dimethyl-4-hydroxy-5-nonanone, 2,8-dimethyl-5-hydroxy-4-nonanone, 2,7-dimethylnonane, 3,8-dimethyl-3-nonene, 3,8-dimethyl-4-nonene, 3,8-dimethyl-5-nonene, 3,8-dimethyl-4-nonanol, 3,8-dimethyl-5-nonanol, 3,8-dimethyl-4-nonanone, 3,8-dimethyl-5-nonanone, 3,8-dimethyl-4,5-nonanediol, 3,8-dimethyl-4,5-nonanedione, 3,8-dimethyl-4-hydroxy-5-nonanone, 3,8-dimethyl-5-hydroxy-4-nonanone, n-decane, 1-decene, 1-decanol, decanoate, 2,9-dimethyldecane, 2,9-dimethyl-3-decene, 2,9-dimethyl-4-decene, 2,9-dimethyl-5-decanol, 2,9-dimethyl-5-decanone, 2,9-dimethyl-5,6-decanediol, 2,9-dimethyl-6-hydroxy-5-decanone, 2,9-dimethyl-5,6-decanedionen-undecane, 1-undecene, 1-undecanol, undecanal, undecanoate, n-dodecane, 1-dodecene, 1-dodecanol, dodecanal, dodecanoate, n-dodecane, 1-decadecene, 1-dodecanol, ddodecanal, dodecanoate, n-tridecane, 1-tridecene, 1-tridecanol, tridecanal, tridecanoate, n-tetradecane, 1-tetradecene, 1-tetradecanol, tetradecanal, tetradecanoate, n-pentadecane, 1-pentadecene, 1-pentadecanol, pentadecanal, pentadecanoate, n-hexadecane, 1-hexadecene, 1-hexadecanol, hexadecanal, hexadecanoate, n-heptadecane, 1-heptadecene, 1-heptadecanol, heptadecanal, heptadecanoate, n-octadecane, 1-octadecene, 1-octadecanol, octadecanal, octadecanoate, n-nonadecane, 1-nonadecene, 1-nonadecanol, nonadecanal, nonadecanoate, eicosane, 1-eicosene, 1-eicosanol, eicosanal, eicosanoate, 3-hydroxy propanal, 1,3-propanediol, 4-hydroxybutanal, 1,4-butanediol, 3-hydroxy-2-butanone, 2,3-butandiol, 1,5-pentane diol, homocitrate, homoisocitorate, b-hydroxy adipate, glutarate, glutarsemialdehyde, glutaraldehyde, 2-hydroxy-1-cyclopentanone, 1,2-cyclopentanediol, cyclopentanone, cyclopentanol, (S)-2-acetolactate, (R)-2,3-Dihydroxy-isovalerate, 2-oxoisovalerate, isobutyryl-CoA, isobutyrate, isobutyraldehyde, 5-amino pentaldehyde, 1,10-diaminodecane, 1,10-diamino-5-decene, 1,10-diamino-5-hydroxydecane, 1,10-diamino-5-decanone, 1,10-diamino-5,6-decanediol, 1,10-diamino-6-hydroxy-5-decanone, phenylacetoaldehyde, 1,4-diphenylbutane, 1,4-diphenyl-1-butene, 1,4-diphenyl-2-butene, 1,4-diphenyl-2-butanol, 1,4-diphenyl-2-butanone, 1,4-diphenyl-2,3-butanediol, 1,4-diphenyl-3-hydroxy-2-butanone, 1-(4-hydeoxyphenyl)-4-phenylbutane, 1-(4-hydeoxyphenyl)-4-phenyl-1-butene, 1-(4-hydeoxyphenyl)-4-phenyl-2-butene, 1-(4-hydeoxyphenyl)-4-phenyl-2-butanol, 1-(4-hydeoxyphenyl)-4-phenyl-2-butanone, 1-(4-hydeoxyphenyl)-4-phenyl-2,3-butanediol, 1-(4-hydeoxyphenyl)-4-phenyl-3-hydroxy-2-butanone, 1-(indole-3)-4-phenylbutane, 1-(indole-3)-4-phenyl-1-butene, 1-(indole-3)-4-phenyl-2-butene, 1-(indole-3)-4-phenyl-2-butanol, 1-(indole-3)-4-phenyl-2-butanone, 1-(indole-3)-4-phenyl-2,3-butanediol, 1-(indole-3)-4-phenyl-3-hydroxy-2-butanone, 4-hydroxyphenylacetoaldehyde, 1,4-di(4-hydroxyphenyl)butane, 1,4-di(4-hydroxyphenyl)-1-butene, 1,4-di(4-hydroxyphenyl)-2-butene, 1,4-di(4-hydroxyphenyl)-2-butanol, 1,4-di(4-hydroxyphenyl)-2-butanone, 1,4-di(4-hydroxyphenyl)-2,3-butanediol, 1,4-di(4-hydroxyphenyl)-3-hydroxy-2-butanone, 1-(4-hydroxyphenyl)-4-(indole-3-)butane, 1-(4-hydroxyphenyl)-4-(indole-3)-1-butene, 1-di(4-hydroxyphenyl)-4-(indole-3)-2-butene, 1-(4-hydroxyphenyl)-4-(indole-3)-2-butanol, 1-(4-hydroxyphenyl)-4-(indole-3)-2-butanone, 1-(4-hydroxyphenyl)-4-(indole-3)-2,3-butanediol, 1-(4-hydroxyphenyl-4-(indole-3)-3-hydroxy-2-butanone, indole-3-acetoaldehyde, 1,4-di(indole-3-) butane, 1,4-di(indole-3)-1-butene, 1,4-di(indole-3)-2-butene, 1,4-di(indole-3)-2-butanol, 1,4-di(indole-3)-2-butanone, 1,4-di(indole-3)-2,3-butanediol, 1,4-di(indole-3)-3-hydroxy-2-butanone, succinate semialdehyde, hexane-1,8-dicarboxylic acid, 3-hexene-1,8-dicarboxylic acid, 3-hydroxy-hexane-1,8-dicarboxylic acid, 3-hexanone-1,8-dicarboxylic acid, 3,4-hexanediol-1,8-dicarboxylic acid, 4-hydroxy-3-hexanone-1,8-dicarboxylic acid, fucoidan, iodine, chlorophyll, carotenoid, calcium, magnesium, iron, sodium, potassium, phosphate, lactic acid, acetic acid, formic acid, isoprenoids, and polyisoprenes, including rubber.

The term “fermentation” as used herein has its ordinary meaning as known to those skilled in the art and can include culturing of a microorganism or group of microorganisms in or on a suitable medium for the microorganisms. The microorganisms can be aerobes, anaerobes, facultative anaerobes, heterotrophs, autotrophs, photoautotrophs, photoheterotrophs, chemoautotrophs, and/or chemoheterotrophs. The cellular activity, including cell growth can be growing aerobic, microaerophilic, or anaerobic. The cells can be in any phase of growth, including lag (or conduction), exponential, transition, stationary, death, dormant, vegetative, sporulating, etc.

The term “plant polysaccharide” as used herein has its ordinary meaning as known to those skilled in the art and can comprise one or more carbohydrate polymers of sugars and sugar derivatives as well as derivatives of sugar polymers and/or other polymeric materials that occur in plant matter. Exemplary plant polysaccharides include lignin, cellulose, starch, pectin, and hemicellulose. Others are chitin, sulfonated polysaccharides such as alginic acid, agarose, carrageenan, porphyran, furcelleran and funoran. Generally, the polysaccharide can have two or more sugar units or derivatives of sugar units. The sugar units and/or derivatives of sugar units can repeat in a regular pattern, or otherwise. The sugar units can be hexose units or pentose units, or combinations of these. The derivatives of sugar units can be sugar alcohols, sugar acids, amino sugars, etc. The polysaccharides can be linear, branched, cross-linked, or a mixture thereof. One type or class of polysaccharide can be cross-linked to another type or class of polysaccharide.

The term “fermentable sugars” as used herein has its ordinary meaning as known to those skilled in the art and can include one or more sugars and/or sugar derivatives that can be utilized as a carbon source by the microorganism, including monomers, dimers, and polymers of these compounds including two or more of these compounds. In some cases, the organism can break down these polymers, such as by hydrolysis, prior to incorporating the broken down material. Exemplary fermentable sugars include, but are not limited to glucose, xylose, arabinose, galactose, mannose, rhamnose, cellobiose, lactose, sucrose, maltose, and fructose.

The term “saccharification” as used herein has its ordinary meaning as known to those skilled in the art and can include conversion of plant polysaccharides to lower molecular weight species that can be utilized by the organism at hand. For some organisms, this would include conversion to monosaccharides, disaccharides, trisaccharides, and oligosaccharides of up to about seven monomer units, as well as similar sized chains of sugar derivatives and combinations of sugars and sugar derivatives. For some organisms, the allowable chain-length can be longer (e.g. 8, 9, 10, 11, 12, 13, 14, 15, 16, 17, 18, 19, or 20 monomer units or more) and for some organisms the allowable chain-length can be shorter (e.g. 1, 2, 3, 4, 5, 6 monomer units).

The term “biomass” as used herein has its ordinary meaning as known to those skilled in the art and can include one or more biological material that can be converted into a biofuel, chemical or other product. One exemplary source of biomass is plant matter. Plant matter can be, for example, woody plant matter, non-woody plant matter, cellulosic material, lignocellulosic material, hemicellulosic material, carbohydrates, pectin, starch, inulin, fructans, glucans, corn, corn stover, sugar cane, grasses, switchgrass, bamboo, algae, crambe, coconut, jatropha, jute and material derived from these. Plant matter can be further described by reference to the chemical species present, such as proteins, polysaccharides and oils. Polysaccharides include polymers of various monosaccharides and derivatives of monosaccharides including glucose, fructose, lactose, galacturonic acid, rhamnose, etc. Plant matter also includes agricultural waste byproducts or side streams such as pomace, corn steep liquor, corn steep solids, corn stover, corn stillage, corn cobs, corn grain, distillers grains, distillers solutes, bagasse, distillers grains, peels, pits, fermentation waste, wood chips, saw dust, wood flour, wood pulp, paper pulp, paper pulp waste steams straw, lumber, sewage, seed cake, husks, rice hulls, leaves, grass clippings, corn stover, (corn grind), and food leftovers. These materials can come from farms, aquatic environments, forestry, industrial sources, households, etc. Another non-limiting example of biomass is animal matter, including, for example milk, meat, fat, bone meal, animal processing waste, and animal waste. “Feedstock” is frequently used to refer to biomass being used for a process, such as those described herein.

The term “medium” as used herein has its ordinary meaning as known to those skilled in the art and can include the entire contents of the combination of soluble and insoluble matter, suspended matter, cells and medium, such as for example the entire contents of a fermentation reaction can be referred to as a fermentation medium.

The term “productivity” as used herein has its ordinary meaning as known to those skilled in the art and can include the mass of a material of interest produced in a given time in a given volume. Units can be, for example, grams per liter-hour, or some other combination of mass, volume, and time. In fermentation, productivity is frequently used to characterize how fast a product can be made within a given fermentation volume. The volume can be referenced to the total volume of the fermentation vessel, the working volume of the fermentation vessel, or the actual volume of medium being fermented. The context of the phrase will indicate the meaning intended to one of skill in the art. Productivity (e.g. g/L/d) is different from “titer” (e.g. g/L) in that productivity includes a time term, and titer is analogous to concentration.

The term “biocatalyst” as used herein has its ordinary meaning as known to those skilled in the art and can include one or more enzymes and/or microorganisms, including solutions, suspensions, and mixtures of enzymes and microorganisms. In some contexts this word will refer to the possible use of either enzymes or microorganisms to serve a particular function, in other contexts the word will refer to the combined use of the two, and in other contexts the word will refer to only one of the two. The context of the phrase will indicate the meaning intended to one of skill in the art.

The terms “conversion efficiency” or “yield” as used herein have their ordinary meaning as known to those skilled in the art and can include the mass of product made from a mass of substrate. The term can be expressed as a percentage yield of the product from a starting mass of substrate. For the production of ethanol from glucose, the net reaction is generally accepted as:

C₆H₁₂O₆→2C₂H₅OH+2CO₂

and the theoretical maximum conversion efficiency or yield is 51% (wt.). Frequently, the conversion efficiency will be referenced to the theoretical maximum, for example, “80% of the theoretical maximum.” In the case of conversion of glucose to ethanol, this statement would indicate a conversion efficiency of 41% (wt.). The context of the phrase will indicate the substrate and product intended to one of skill in the art. For substrates comprising a mixture of different carbon sources such as found in biomass (xylan, xylose, glucose, cellobiose, arabinose cellulose, hemicellulose etc.), the theoretical maximum conversion efficiency of the biomass to ethanol is an average of the maximum conversion efficiencies of the individual carbon source constituents weighted by the relative concentration of each carbon source. In some cases, the theoretical maximum conversion efficiency is calculated based on an assumed saccharification efficiency. By way of example only, given carbon source comprising 10 g of cellulose, the theoretical maximum conversion efficiency can be calculated by assuming saccharification of the cellulose to the assimilable carbon source glucose of about 75% by weight. In this example, 10 g of cellulose can provide 7.5 g of glucose which can provide a maximum theoretical conversion efficiency of about 7.5 g*51% or 3.8 g of ethanol. In other cases, the efficiency of the saccharification step can be calculated or determined (i.e. measured). Saccharification efficiencies anticipated by the present invention include about 10%, 20%, 30%, 40%, 50%, 60%, 70%, 80%, 90%, 95%, 99% or about 100% for any carbohydrate carbon sources larger than a single monosaccharide subunit.

“Pretreatment” or “pretreated” is used herein to refer to any mechanical, chemical, thermal, biochemical process or combination of these processes whether in a combined step or performed sequentially, that achieves disruption or expansion of the biomass so as to render the biomass more susceptible to attack by enzymes and/or microbes. In some embodiments, pretreatment can include removal or disruption of lignin so is to make the cellulose and hemicellulose polymers in the plant biomass more available to cellulolytic enzymes and/or microbes, for example, by treatment with acid or base. In some embodiments, pretreatment can include the use of a microorganism of one type to render plant polysaccharides more accessible to microorganisms of another type. In some embodiments, pretreatment can also include disruption or expansion of cellulosic and/or hemicellulosic material. Steam explosion, and ammonia fiber expansion (or explosion) (AFEX) are well known thermal/chemical techniques. Hydrolysis, including methods that utilize acids and/or enzymes can be used. Other thermal, chemical, biochemical, enzymatic techniques can also be used.

The term “fed-batch” or “fed-batch fermentation” as used herein has its ordinary meaning as known to those skilled in the art and can include a method of culturing microorganisms where nutrients, other medium components, or biocatalysts (including, for example, enzymes, fresh organisms, extracellular medium, etc.) are supplied to the fermentor during cultivation, but culture medium is not harvested from the fermentor until the end of the fermentation, although it can also include “self seeding” or “partial harvest” techniques where a portion of the fermentor volume is harvested and then fresh medium is added to the remaining medium in the fermentor, with at least a portion of the inoculum being the medium that was left in the fermentor. In some embodiments, a fed-batch process might be referred to with a phrase such as, “fed-batch with cell augmentation.” This phrase can include an operation where nutrients and microbial cells are added or one where microbial cells with no substantial amount of nutrients are added. The more general phrase “fed-batch” encompasses these operations as well. The context where any of these phrases is used will indicate to one of skill in the art the techniques being considered.

A term “phytate” as used herein has its ordinary meaning as known to those skilled in the art can be include phytic acid, its salts, and its combined forms as well as combinations of these.

The term “sugar compounds” as used herein has its ordinary meaning as known to those skilled in the art and can include monosaccharide sugars, including but not limited to hexoses and pentoses; sugar alcohols; sugar acids; sugar amines; compounds containing two or more of these linked together directly or indirectly through covalent or ionic bonds; and mixtures thereof. Included within this description are disaccharides; trisaccharides; oligosaccharides; polysaccharides; and sugar chains, branched and/or linear, of any length.

a. Introduction

Biomass is a renewable source of energy, which can be biologically fermented to produce an end-product such as a fuel (e.g. alcohol, ethanol, organic acid, acetic acid, lactic acid, methane, or hydrogen) portable for mobile engines or a chemical compound for other commercial purposes. Biomass includes agricultural residues (corn stalks, grass, straw, grain hulls, bagasse, etc.), animal waste (manure from cattle, poultry, and hogs), algae, woody materials (wood or bark, sawdust, timber slash, and mill scrap), municipal waste (waste paper, recycled toilet papers, yard clippings, etc.), and energy crops (poplars, willows, switchgrass, alfalfa, prairie bluestem, etc.). Lignocellulosic biomass has cellulose and hemicellulose as two major components. To obtain a high fermentation efficiency of lignocellulosic biomass to end-product (yield) it can be important to provide an appropriate pretreatment for removing and/or detoxifying at least a portion of the lignin content and for making cellulose and hemicelluloses more amendable to enzymatic hydrolysis.

Recently, the conversion to ethanol of polymeric hexose and pentose sugars in cellulose and hemicellulose has been achieved. See U.S. Pat. No. 4,349,628 to English et al; see also U.S. Pat. No. 4,400,470 to Zeikus et at; U.S. Pat. No. 5,000,000 to Ingram et al; U.S. Pat. No. 5,028,539 to Ingram et al; and U.S. Pat. No. 5,162,516 to Ingram et al, all of which are incorporated herein by reference.

In some embodiments, fuel production from biomass is a two step process involving enzymatic hydrolysis followed by fermentation. Enzymatic hydrolysis of biomass can be achieved using commercially available hydrolytic enzyme cocktails, enzymes derived from a specific organism or group of organisms, spent fermentation medium, or any source of carbohydrate degrading or saccharifying enzymes. In one embodiment an enzyme useful for the treatment of biomass includes, but is not limited to a xylanases, endo-1,4-beta-xylanases, xylosidases, beta-D-xylosidases, cellulases, hemicellulases, carbohydrases, glucanases, endoglucanases, endo-1,4-beta-glucanases, exoglucanases, glucosidases, beta-D-glucosidases, amylases, cellobiohydrolases, exocellobiohydrolases, phytases, proteases, pectate lyases, galacturonases, laccases amylase, protease, chitinase, pectinase, or a keratinase.

Additional methods and compositions for treatment of biomass, pretreatment of biomass, enzymatic treatment of biomass, or preparation of biomass for fermentation or conversion to useful end-products are provided by US Patent Application Nos. 20090053770, 20070031918, 20070031953, 20090053777, 20090042259, 20090042266, 20090004698, 20090004692, 20090004706, 20090011474, 20090011484, 20080227161, 20080227162, 20080044877, 20080182323, 20070148751, 20060246563, and U.S. Pat. Nos. 5,865,898, 5,628,830, 5,693,296, 5,837,506, and 6090595 each of which are herein incorporated by reference in their entirety. Enzyme treatment of biomass results in degradation of high molecular weight carbohydrate polymers into smaller oligosaccharides and in some cases, eventually, monomeric hexose and pentose sugars. In the second step these sugars are fermented to an end-product (e.g. alcohol, ethanol, organic acid, acetic acid, lactic acid, methane, or hydrogen) using, for example, yeast or bacterial strains.

In other embodiments, the hydrolysis and fermentation process can be combined into a single step. In some cases, this can provide a process that is more economical than a two step process by reducing capital and operational costs, for example, by minimizing the need for external enzyme treatment. In still other embodiments, conversion of both hexose and pentose sugars to end-products can provide enhanced yields of end-products per gram of biomass as compared to conversion of only hexoses or only pentoses, or as compared to processes which mainly convert pentoses but do not substantially convert hexoses or mainly convert hexoses but do not substantially convert pentoses. Commonly used species of yeast (Saccharomyces cerevisiae), fungi and bacteria have been reported to be able to readily convert hexose sugar (glucose) to ethanol. However, fermentation of pentose sugars (xylose and arabinose) is still a technological bottleneck for ethanol production from biomass. Some of the researchers have used genetic tools to obtain recombinants of Zymomonas, E. coli, Saccharomyces and other yeasts.

The present invention provides methods for use of microorganisms, such as Clostridium phytofermentans or other Clostridium species, which in some embodiments have the capability of simultaneously hydrolyzing and fermenting lignocellulosic biomass. In one embodiment a microorganism simultaneously ferments both hexose and pentose fractions to produce a fermentation end-product. In another embodiment Clostridium phytofermentans or other Clostridium species can provide useful advantages for the conversion of biomass to ethanol or other fermentation end-products (e.g. alcohol, organic acid, acetic acid, lactic acid, methane, or hydrogen) by its ability to produce enzymes capable of hydrolyzing polysaccharides and higher saccharides to lower molecular weight saccharides, oligosaccharides, disaccharides, and monosaccharides. In some embodiments, a microorganism (such as Clostridium phytofermentans or other Clostridium species) can be utilized in methods described herein to perform the combined steps of hydrolyzing a higher molecular weight biomass containing sugars and/or higher saccharides or polysaccharides to lower sugars and fermenting oligosaccharides, disaccharides, and monosaccharides from both cellulose as well as hemicelluloses into one or more fermentation end-products (including, but not limited to ethanol, methane, hydrogen, and other compounds such as organic acids including formic acid, acetic acid, and lactic acid). Methods described herein further provide for the growth, culturing, fermenting etc. of a microorganism, such as Clostridium phytofermentans or another Clostridium species under conditions that include elevated ethanol concentration, high sugar concentration, low sugar concentration, utilization of insoluble carbon sources, and anaerobic conditions.

In one embodiment Clostridium phytofermentans or another Clostridium species preferentially ferments oligomers instead of monosaccharides. This metabolic trait can be utilized to reduce the time and severity of pretreatment of biomass. For example, a less severe acid treatment resulting in the release of oligomers rather than monosaccharides reduces the time and cost of chemicals of the pretreatment process. This can result in lower overall cost of producing a fermentation end-product. Fewer sugars are degraded during such a process thus adding to the higher saccharide content of the biomass and an increased yield of ethanol or other chemical product.

In some embodiments, the methods of the present invention provide for a fermentation process, such as for example a continuous fermentation process, a batch fermentation process, or a fed-batch fermentation process (e.g. constant or variable volume). In some embodiments, the methods provide for the fermentation of biomass with a microorganism, such as Clostridium phytofermentans or another Clostridium species. In some cases, a fed-batch fermentation process is provided for ethanol production from biomass (e.g. corn stover or any biomass provided herein) using a microorganism, such as Clostridium phytofermentans or another Clostridium species. In one embodiment a method provides for titers of 5 to 200 g/L of ethanol with a production rate of about 0.5 to 20 g/L/d. In another embodiment a method provides for an ethanol yield of about 0.1-1 grams ethanol per gram of biomass loaded in the fermentor. In some embodiments, a method provides for yields of about 45%-99.5%, or more of the theoretical maximum possible yield of fermentation end product (e.g. alcohol, ethanol, organic acid, acetic acid, lactic acid, methane, or hydrogen).

In one embodiment a method provides for titers of at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 4, 142, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120 125, 130, 135, 140, 145, 150, 160, 170, 180, 190, or 200 g/L or more of ethanol with a production rate of about 0.5, 0.6, 0.7, 0.8, 0.9, 1, 2, 3, 4, 5, 6, 7, 8, 9, 10, 11, 12, 13, 14, 15, 16, 17, 18, 19, or g/L/d or more. In another embodiment a method provides for an ethanol yield of about 0.1, 0.2, 0.3, 0.4, 0.5, 0.6, 0.7, 0.8, 0.9, or 1 g or more ethanol per g of biomass loaded in the fermentor. In some embodiments, a method provides for yields of at least about 50%, 60%, 75%, 80%, 85%, 90%, 91%, 92%, 93%, 94%, 95%, 96%, 97%, 98%, 99%, 99.5%, or more of the theoretical maximum possible yield of fermentation end product (e.g. alcohol, ethanol, organic acid, acetic acid, lactic acid, methane, or hydrogen).

b. Methods

### Fermentation

The following description and examples illustrate certain preferred embodiments of the present invention in detail. Those of skill in the art will recognize that there are numerous variations and modifications that are encompassed by its scope. Accordingly, the description of a preferred embodiment should not be deemed to limit the scope of the present invention.

Methods are provided herein for the fermentation of biomass and the subsequent production of a useful end-product including, but not limiting to, an alcohol, ethanol, an organic acid, acetic acid, lactic acid, methane, or hydrogen or other chemical. In some embodiments, biomass (e.g. corn stover) is processed or pretreated prior to fermentation. In one embodiment a method of pre-treatment includes but is not limited to, biomass particle size reduction, such as for example shredding, milling, chipping, crushing, grinding, or pulverizing. In some embodiments, biomass particle size reduction can include size separation methods such as sieving, or other suitable methods known in the art to separate materials based on size. In one embodiment size separation can provide for enhanced yields. In some embodiments, separation of finely shredded biomass (e.g. particles smaller than about 8 mm in diameter, such as, 8, 7.9, 7.7, 7.5, 7.3, 7, 6.9, 6.7, 6.5, 6.3, 6, 5.9, 5.7, 5.5, 5.3, 5, 4.9, 4.7, 4.5, 4.3, 4, 3.9, 3.7, 3.5, 3.3, 3, 2.9, 2.7, 2.5, 2.3, 2, 1.9, 1.7, 1.5, 1.3, 1, 0.9, 0.8, 0.7, 0.6, 0.5, 0.4, 0.3, 0.2, or 0.1 mm) from larger particles allows the recycling of the larger particles back into the size reduction process, thereby increasing the final yield of processed biomass.

In some embodiments pretreatment methods can include treatment under conditions of high or low pH. High or low pH treatment includes, but is not limited to, treatment using concentrated acids or concentrated alkali, or treatment using dilute acids or dilute alkali. Alkaline compositions useful for treatment of biomass in the methods of the present invention include, but are not limited to, caustic, such as caustic lime, caustic soda, caustic potash, sodium, potassium, or calcium hydroxide, or calcium oxide. In some embodiments suitable amounts of alkaline useful for the treatment of biomass ranges from 0.01 g to 3 g of alkaline (e.g. caustic) for every gram of biomass to be treated. In some embodiments suitable amounts of alkaline useful for the treatment of biomass include, but are not limited to, about 0.01 g of alkaline (e.g. caustic), 0.02 g, 0.03 g, 0.04 g, 0.05 g, 0.075 g, 0.1 g, 0.2 g, 0.3 g, 0.4 g, 0.5 g, 0.75 g, 1 g, 2 g, or about 3 g of alkaline (e.g. caustic) for every gram of biomass to be treated.

In another embodiment, biomass can be pre-treated at an elevated temperature and/or pressure. In one embodiment biomass is pre treated at a temperature range of 20° C. to 400° C. In another embodiment biomass is pretreated at a temperature of about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C., 350° C., 400° C. or higher. In another embodiment, elevated temperatures are provided by the use of steam, hot water, or hot gases. In one embodiment steam can be injected into a biomass containing vessel. In another embodiment the steam, hot water, or hot gas can be injected into a vessel jacket such that it heats, but does not directly contact the biomass.

In another embodiment, a biomass can be treated at an elevated pressure. In one embodiment biomass is pre treated at a pressure range of about 1 psi to about 30 psi. In another embodiment biomass is pre treated at a pressure or about 1 psi, 2 psi, 3 psi, 4 psi, 5 psi, 6 psi, 7 psi, 8 psi, 9 psi, 10 psi, 12 psi, 15 psi, 18 psi, 20 psi, 22 psi, 24 psi, 26 psi, 28 psi, 30 psi or more. In some embodiments, biomass can be treated with elevated pressures by the injection of steam into a biomass containing vessel. In other embodiments, the biomass can be treated to vacuum conditions prior or subsequent to alkaline or acid treatment or any other treatment methods provided herein.

In one embodiment alkaline or acid pretreated biomass is washed (e.g. with water (hot or cold) or other solvent such as alcohol (e.g. ethanol)), pH neutralized with an acid, base, or buffering agent (e.g. phosphate, citrate, borate, or carbonate salt) or dried prior to fermentation. In one embodiment, the drying step can be performed under vacuum to increase the rate of evaporation of water or other solvents. Alternatively, or additionally, the drying step can be performed at elevated temperatures such as about 20° C., 25° C., 30° C., 35° C., 40° C., 45° C., 50° C., 55° C., 60° C., 65° C., 80° C., 90° C., 100° C., 120° C., 150° C., 200° C., 250° C., 300° C. or more.

In some embodiments of the present invention, the pretreatment step includes a step of solids recovery. The solids recovery step can be during or after pretreatment (e.g., acid or alkali pretreatment), or before the drying step. In some embodiments, the solids recovery step provided by the methods of the present invention includes the use of a sieve, filter, screen, or a membrane for separating the liquid and solids fractions. In one embodiment a suitable sieve pore diameter size ranges from about 0.001 microns to 8 mm, such as about 0.005 microns to 3 mm or about 0.01 microns to 1 mm. In one embodiment a sieve pore size has a pore diameter of about 0.01 microns, 0.02 microns, 0.05 microns, 0.1 microns, 0.5 microns, 1 micron, 2 microns, 4 microns, 5 microns, 10 microns, 20 microns, 25 microns, 50 microns, 75 microns, 100 microns, 125 microns, 150 microns, 200 microns, 250 microns, 300 microns, 400 microns, 500 microns, 750 microns, 1 mm or more.

In one embodiment, fed-batch fermentation is performed on the treated biomass to produce a fermentation end-product, such as alcohol, ethanol, organic acid, acetic acid, lactic acid, methane, or hydrogen. In one embodiment, the fermentation process comprises simultaneous hydrolysis and fermentation of the biomass using one or more microorganisms such as a Clostridium strain, a Trichoderma strain, a Saccharomyces strain, a Zymomonas strain, or another microorganism suitable for fermentation of biomass. In another embodiment, the fermentation process comprises simultaneous hydrolysis and fermentation of the biomass using a microorganism that is Clostridium phytofermentans, Clostridium algidixylanolyticum, Clostridium xylanolyticum, Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Acetivibrio cellulolyticus, Bacteroides cellulosolvens, Caldicellulosiruptor saccharolyticum, Ruminococcus albus, Ruminococcus flavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum, Sacharophagus degradans, or Thermoanaerobacterium saccharolyticum.

In one embodiment, one or more microorganisms used for the fermentation of biomass is a wild-type microorganism. Wild-type microorganisms are those microorganisms that are substantially similar to an isolate obtained from a natural environment and include isolates that have been propagated in a laboratory environment. In another embodiment, one or more of the microorganisms is bred and/or selected for a desirable trait. Methods of selection or breeding can include growth with a medium comprising a carbon source that is or approximates the carbon source to be utilized in the production of fermentation end-products. Desirable traits include but are not limited to increased biomass saccharification, increased production of a specific fermentation end-product, increased ethanol production, increased tolerance to ethanol, increased enzyme synthesis or decreased sporulation. A method of selection can further include the use of mutagenesis (e.g. by chemical or irradiation means) to generate a desired populations of microorganisms. Mutagenized microorganisms can then be selected for desired traits, leading to a higher frequency of desirable isolates. In another embodiment, one or more microorganisms used for fermenation can be a recombinant microorganism. A Recombinant microorganism comprises one or more changes to its nucleic acids in comparison to a respective wild-type microorganism that did not arise by spontaneous (i.e. natural) mutation. In one embodiment a recombinant microorganisms comprises one an exogenous polynucleic acid from another species (such as another microorganism), a synthetic polynucleic acid, or a polynucleic acid isolated or derived from the same species.

In one embodiment, the fermentation process can include simultaneous hydrolysis and fermentation of a biomass with one or more enzymes, such as a xylanases, endo-1,4-beta-xylanases, xylosidases, beta-D-xylosidases, cellulases, hemicellulases, carbohydrases, glucanases, endoglucanases, endo-1,4-beta-glucanases, exoglucanases, glucosidases, beta-D-glucosidases, amylases, cellobiohydrolases, exocellobiohydrolases, phytases, proteases, peroxidase, pectate lyases, galacturonases, or laccases. In one embodiment one or more enzymes used to treat a biomass is thermostable. In another embodiment a biomass is treated with one or more enzymes, such as those provided herein, prior to fermentation. In another embodiment a biomass is treated with one or more enzymes, such as those provided herein, during fermentation. In another embodiment a biomass is treated with one or more enzymes, such as those provided herein, prior to fermentation and during fermentation. In another embodiment an enzyme used for pretreatment of a biomass is the same as those added during fermentation. In another embodiment an enzyme used for pretreatement of biomass is different from those added during fermentation.

In some embodiments, fermentation can be performed in an apparatus such as bioreactor, a fermentation vessel, a stirred tank reactor, or a fluidized bed reactor. In one embodiment the treated biomass can be supplemented with suitable chemicals to facilitate robust growth of the one or more fermenting organisms. In one embodiment a useful supplement includes but is not limited to, a source of nitrogen and/or amino acids such as yeast extract, cysteine, or ammonium salts (e.g. nitrate, sulfate, phosphate etc.); a source of simple carbohydrates such as corn steep liquor, and malt syrup; a source of vitamins such as yeast extract; buffering agents such as salts (including but not limited to citrate salts, phosphate salts, or carbonate salts); or mineral nutrients such as salts of magnesium, calcium, or iron. In some embodiments redox modifiers are added to the fermentation mixture including but not limited to cysteine or mercaptoethanol.

Chemicals used in the methods of the present invention are readily available and can be purchased from a commercial supplier, such as Sigma-Aldrich. Additionally, commercial enzyme cocktails (e.g. Accellerase™ 1000, CelluSeb-TL, CelluSeb-TS, Cellic™ CTec, STARGEN™, Maxalig™, Spezyme®, Distillase®, G-Zyme®, Fermenzyme®, Fermgen™, GC 212, or Optimash™) or any other commercial enzyme cocktail can be purchased from vendors such as Specialty Enzymes & Biochemicals Co., Genencor, or Novozymes. Alternatively, enzyme cocktails can be prepared by growing one or more organisms such as for example a fungi (e.g. a Trichoderma, a Saccharomyces, a Pichia, a White Rot Fungus etc.), a bacteria (e.g. a Clostridium (e.g. Clostridium phytofermentans), or a coliform bacterium, a Zymomonas bacterium, Sacharophagus degradans etc.) in a suitable medium and harvesting enzymes produced therefrom. In some embodiments, the harvesting can include one or more steps of purification of enzymes.

In one embodiment the titer and/or productivity of fermentation end-product production by a microorganism (such as Clostridium phytofermentans) is improved by culturing the microorganism in a medium comprising one or more compounds comprising hexose and/or pentose sugars. In one embodiment, a process comprises conversion of a starting material (such as a biomass) to a biofuel, such as one or more alcohols. In one embodiment, methods of the invention comprise contacting substrate comprising both hexose (e.g. glucose, cellobiose) and pentose (e.g. xylose, arabinose) saccharides with a microorganism that can hydrolyse C5 and C6 saccharides to produce ethanol. In another embodiment, methods of the invention comprise contacting substrate comprising both hexose (e.g. glucose, cellobiose) and pentose (e.g. xylose, arabinose) saccharides with C. phytofermentans to produce ethanol.

In some embodiments of the present invention, batch fermentation with a microorganism (such as Clostridium phytofermentans) of a mixture of hexose and pentose saccharides using the methods of the present invention provides uptake rates of about 0.1-8 g/L/h or more of hexose (e.g. glucose, cellulose, cellobiose etc.), and about 0.1-8 g/L/h or more of pentose (xylose, xylan, hemicellulose etc.). In some embodiments of the present invention, batch fermentation with a microorganism (such as Clostridium phytofermentans) of a mixture of hexose and pentose saccharides using the methods of the present invention provides uptake rates of about 0.1, 0.2, 0.4, 0.5, 0.6 0.7, 0.8, 1, 2, 3, 4, 5, or 6 g/L/h or more of hexose (e.g. glucose, cellulose, cellobiose etc.), and about 0.1, 0.2, 0.4, 0.5, 0.6 0.7, 0.8, 1, 2, 3, 4, 5, or 6 g/L/h or more of pentose (xylose, xylan, hemicellulose etc.).

In one embodiment a method for production of ethanol produces about 10 g/l to 120 gain 40 hours or less. In another embodiment a method for production of ethanol produces about 10 g/l, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, 25 g/L, 26 g/L, 27 g/L, 28 g/L, 29 g/L, 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L, 86 g/L, 87 g/L, 88 g/L, 89 g/L, 90 g/L, 91 g/L, 92 g/L, 93 g/L, 94 g/L, 95 g/L, 96 g/L, 97 g/L, 98 g/L, 99 g/L, 100 g/L, 110 g/l, 120 g/l, or more ethanol in 40 hours by the fermentation of biomass. In another embodiment, ethanol produced by a method comprising simultaneous fermentation of hexose and pentose saccharides. In another embodiment, ethanol is produced to by a microorganism comprising simultaneous fermentation of hexose and pentose saccharides.

In another embodiment a microorganism that produces a fermentative end-product tolerates in the presence of high alcohol (e.g. ethanol or butanol) concentrations. In one embodiment Clostridium phytofermentans tolerates in the presence of high alcohol (e.g. ethanol or butanol) concentrations. In one embodiment the microorganism can grow and function in alcohol (e.g. ethanol or butanol) concentrations up 15% v/v. In another embodiment the microorganism can grow and function in alcohol (e.g. ethanol or butanol) concentrations of 1%, 2%, 3%, 4%, 5%, 6%, 7%, 8%, 9%, 10%, 11%, 12%, 13%, 14%, or 15% v/v. In one embodiment functioning in high alcohol concentrations includes the ability to continue to produce alcohol without undue inhibition or suppression by alcohol and/or other components present. In another embodiment functioning in high alcohol concentrations includes the ability to efficiently convert hexose and pentose carbon sources in a biomass feedstock to a fermentation end-product such as an alcohol. In one embodiment Clostridium phytofermentans tolerates in the presence of high alcohol (e.g. ethanol or butanol) concentrations.

It has been observed that an ethanol concentration in a fermentation medium comprising Clostridium phytofermentans attains a plateau of about 15 g/L after about 36-48 hours of batch fermentation, with carbon substrate remaining in the medium. In another embodiment lowering the fermentation pH to about 6.5 and/or adding unsaturated fatty acids to the fermentation medium resulted in a significant increase in the amount of ethanol produced by the organism, with between about 20 g/L to about 30, 40, 50, 60, or 70 g/L or more of ethanol observed in the medium following a 48-96 hrs or longer fermentation. In addition, it has also been observed that the productivity of the organism was higher (to about 10 g/L-d) when the ethanol titer was low and lower (to about 2 g/L-d) than when the ethanol concentration was higher. Fermentation at reduced pH and/or with the addition of a lipid (e.g., fatty acids) can result in about a two to ten fold (such as a 2×, 3×, 4×, 5×, 6×, 7×, 8×, 9×, or 10× increase) or higher increase in the ethanol production rate as compared to the unadjusted fermentation medium. In some embodiments of the present invention, simultaneous fermentation of both hexose and pentose saccharides can also enable increases in ethanol productivity and/or yield. In some cases, the simultaneous fermentation of hexose and pentose carbohydrate substrates can be utilized in combination with fermentation at reduced pH and/or with the addition of a lipid (e.g., fatty acids) to further increase productivity, and/or yield. In one embodiment a lipid is a fat or oil, including without limitation the glyceride esters of fatty acids along with associated phosphatides, sterols, alcohols, hydrocarbons, ketones, and related compounds. In another embodiment a lipid is a phospholipid. In one embodiment a fatty acid is an aliphatic or aromatic monocarboxylic acid. In another embodiment a fatty acid is an unsaturated fatty acid. In one embodiment an unsaturated fatty acid is a fatty acid with 1 to 3 double bonds and a “highly unsaturated fatty acid” means a fatty acid with 4 or more double bonds. In another embodiment an unsaturated fatty acid is a omega-3 highly unsaturated fatty acid, such as eicosapentaenoic acid, docosapentaenoic acid, alpha linolenic acid, docosahexaenoic acid, and conjugates thereof. In another embodiment a fatty acid is a saturated fatty acid. In another embodiment a fatty acid is a vegetable oil, such as partially hydrogenated, include palm oil, cottonseed oil, corn oil, peanut oil, palm kernel oil, babassu oil, sunflower oil, safflower oil, or mixtures thereof. In another embodiment a composition comprising a fatty acid further comprises a wax, such as beeswax, petroleum wax, rice bran wax, castor wax, microcrystalline wax, or mixtures thereof.

In another embodiment a biomass is pre-treated with a surfactant prior to fermentation with a microorganism. In another embodiment a biomass is contacted with a surfactant during fermentation with a microorganism. In one embodiment the surfactant is a Tween series of surfactant (e.g., Tween 20 or Tween 80) or a Triton series of surfactant (e.g. Triton X-100). In another embodiment the surfactant is polysorbate 60, polysorbate 80, propylene glycol, sodium dioctylsulfoesuccinate, sodium lauryl sulfate, lactylic esters of fatty acids, polyglycerol esters of fatty acids, or mixtures thereof. In another embodiment a biomass is pre-treated with a surfactant and a lipid prior to fermentation with a microorganism. In another embodiment a biomass is contacted with a surfactant and a lipid during fermentation with a microorganism.

In another embodiment the fermentation medium comprises a chelating agent (such as the dihydrate of trisodium citrate, or EDTA). In one embodiment a chelating agent is a chemical that forms soluble, complex molecules with certain metal ions, inactivating the ions so that they do not react with other elements or ions. In one embodiment, the concentration of a chelating agent in the fermentation medium is greater than about 0.2 g/L, greater than about 0.5 g/L, or greater than about 1 g/L. In another embodiment, the concentration of a chelating agent in the fermentation medium is less than about 10 g/L, less than about 5 g/L, or less than about 2 g/L. In one embodiment a biologically acceptable chelating agent is 5-Sulfosalicylic acid dihydrate, Ammonium citrate dibasic, Ammonium oxalate monohydrate, Citric acid, Ethylenediaminetetraacetic acid, Ethylenediaminetetraacetic acid disodium salt dihydrate, L-(+)-Tartaric acid, Potassium oxalate monohydrate, Potassium sodium tartrate tetrahydrate, Sodium citrate tribasic dihydrate, Sodium L-tartrate dibasic dihydrate, Sodium oxalate, Ethylene glycol-bis(2-aminoethylether)-N,N,N′,N′-tetraacetic acid, Magnesium citrate tribasic nonahydrate, Ethylenediaminetetraacetic acid diammonium salt, Ethylenediaminetetraacetic acid dipotassium salt dihydrate, Potassium tetraoxalate dihydrate, Sodium tartrate dibasic dihydrate, Ethylenediaminetetraacetic acid tripotassium salt dihydrate, Ethylenediaminetetraacetic acid trisodium salt dihydrate, Ammonium tartrate dibasic, Lithium citrate tribasic tetrahydrate, Potassium citrate monobasic, Sodium bitartrate monohydrate, Sodium citrate monobasic, Ethylenediaminetetraacetic acid tetrasodium salt hydrate, N,N-Dimethyldecylamine N-oxide, N,N-Dimethyldodecylamine N-oxide, Nitrilotriacetic acid, Potassium citrate tribasic, Potassium D-tartrate monobasic, Potassium peroxodisulfate, Potassium sodium tartrate, Pyromellitic acid hydrate, Sodium tartrate dibasic solution, Citrate Concentrated Solution, Ethylenediaminetetraacetic acid disodium salt, Edetate disodium, Sodium citrate, Ethylenediaminetetra(methylenephosphonic acid), dicarboxymethylglutamic acid, ethylenediaminedisuccinic acid (EDDS), methylamine, pyocyanin, pyoverdin, enterobactin, methionine e.g., phytochelatin, malic acid, nitrilotriacetic acid, oxalic acid, or desferrioxamine B. In one embodiment a chelating agent is chosen based on the specificity of the metal(s) targeted for chelation and/or by the ability of the chelating agent to function in the pretreatment environment. In one embodiment, where an alkaline pH is maintained by the addition of an alkaline agent to the feedstock, the chelating agent chosen would be capable of functioning at alkaline pH. In another embodiment, where an acid pH is maintained by the addition of an acid agent to the feedstock, the chelating agent chosen would be capable of functioning at acid pH. In another embodiment, where high temperature is utilized during pretreatment, the chelating agent chosen would be capable of functioning at high temperature. In another embodiment, a fermentation medium comprises more than one chelating agent. In one embodiment one or more chelating agent is added to a fermentation medium during fermentation of a biomass with a microorganism, such as Clostridium phytofermentans.

Biomass

In some embodiments, a microorganism, (such as Clostridium phytofermentans) is contacted with pretreated or non-pretreated feedstock containing cellulosic, hemicellulosic, and/or lignocellulosic material. Additional nutrients can be present or added to the biomass material to be processed by the microorganism including nitrogen-containing compounds such as amino acids, proteins, hydrolyzed proteins, ammonia, urea, nitrate, nitrite, soy, soy derivatives, casein, casein derivatives, milk powder, milk derivatives, whey, yeast extract, hydrolyze yeast, autolyzed yeast, corn steep liquor, corn steep solids, monosodium glutamate, and/or other fermentation nitrogen sources, vitamins, and/or mineral supplements. In some embodiments, one or more additional lower molecular weight carbon sources can be added or be present such as glucose, sucrose, maltose, corn syrup, Distillers Dried Solubles (DDS), Distillers Dried Grains (DDG), Condensed Distillers Solubles (CDS), Distillers Wet Grains (DWG), Distillers Dried Grains with Solubles (DDGS), lactic acid, etc.

Such lower molecular weight carbon sources can serve multiple functions including providing an initial carbon source at the start of the fermentation period, help build cell count, control the carbon/nitrogen ratio, remove excess nitrogen, or some other function. In some embodiments another medium supplement is added, such as pH modifier, a lipid (e.g., a fatty acid), a surfactant or a chelating agent.)

In some embodiments aerobic/anaerobic cycling is used for the bioconversion of cellulosic or lignocellulosic material to fuels and chemicals. In some embodiments, the anaerobic microorganism can ferment biomass directly without the need of a pretreatment. In some embodiments, feedstocks are contacted with biocatalysts capable of breaking down plant-derived polymeric material into lower molecular weight products that can subsequently be transformed by biocatalysts to fuels and/or other desirable chemicals.

In some embodiments, the invention provides for a process for simultaneous saccharification and fermentation of cellulosic solids from biomass into a fuel or another fermentation end-product by a microorganism. In one embodiment the microorganism is Clostridium phytofermentans.

In one embodiment hydrolysis of the pretreated feedstock and hydrolysis of the oligosaccharides by a microorganism occurs simultaneously in a single fermentation reaction vessel. In one embodiment the microorganism is Clostridium phytofermentans. In another embodiment, hydrolysis of a pretreated feedstock, hydrolysis of oligosaccharides and conversion of monosaccharides to ethanol can occur simultaneously in a single reaction vessel. In one embodiment a single microorganism performs both of the hydrolysis and the conversion. In one embodiment the microorganism is Clostridium phytofermentans. In another embodiment a first and second species of microorganisms perform the hydrolysis and the conversion steps.

In one embodiment the process comprises treating the biomass in a closed container with a microorganism that can saccharify C5/C6 saccharides. In another embodiment the process comprises treating the biomass in a closed container with Clostridium phytofermentans bacterium or another Clostridium species under conditions wherein the Clostridium phytofermentans or other microorganism produces saccharolytic enzymes sufficient to substantially convert the biomass into monosaccharides and disaccharides. In another embodiment, the process comprises treating the biomass in a container with a microorganism that can saccharify C5/C6 saccharides and adding one or more enzymes to aid in the breakdown or detoxification of carbohydrates or lignocellulosic material. In another embodiment, the process comprises treating the biomass in a container with a Clostridium phytofermentans or another similar C5/C6 Clostridium species and adding one or more enzymes to aid in the breakdown or detoxification of carbohydrates or lignocellulosic material. In some embodiments, the culture can then be contacted after fermentation with a first microorganism (such as Clostridium phytofermentans) with a second microorganism where the second organism is capable of substantially converting the monosaccharides and disaccharides into a desired fermentation end-product, such as a fuel (e.g. ethanol or butanol). In one embodiment the second microorganisms is a fungi. In another embodiment the second microorganism is a yeast. In another embodiment the second microorganism is Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus. In another embodiment the second microorganism is Saccharomyces or Candidia. In another embodiment the second microorganism is a Clostridia species such as C. thermocellum, C. acetobutylicum, and C. cellovorans, or Zymomonas mobilis.

In some embodiments, a process is provided for producing a biofuel or other chemical from a lignin-containing biomass. The process comprises: 1) contacting the lignin-containing biomass with an aqueous alkaline solution at a concentration sufficient to hydrolyze at least a portion of the lignin-containing biomass; 2) neutralizing the treated biomass to a pH between 5 to 9 (e.g. 5.5, 6, 6.5, 7, 7.5, 8, 8.5, or 9); 3) treating the biomass in a closed container with a Clostridium phytofermentans or another similar C5/C6 Clostridium species bacterium under conditions wherein the Clostridium phytofermentans, optionally with the addition of one or more enzymes to the container, substantially converts the treated biomass into monosaccharides and disaccharides, and/or biofuel or other fermentation end-product; and 4) optionally, introducing a culture of a second microorganism wherein the second organism is capable of substantially converting the monosaccharides and disaccharides into a fermentation end-product, such as a biofuel.

### Genetic Modification of Microorganism

In another aspect, compositions and methods are provided to produce a fermentation end-product such as one or more alcohols, e.g., ethanol, by the creation and use of a genetically modified microorganism. In one embodiment the genetically modified microorganism is Clostridium phytofermentans. In one embodiment the genetic modification is to a nucleic acid sequence that regulates or encodes a protein related to a fermentative biochemical pathways, expression of saccharolytic enzymes, or increasing tolerance of environmental conditions during fermentation. In another embodiment the genetic modification is to a nucleic acid sequence in a microorganism. In one embodiment, the microorganism is transformed with polynucleotides encoding one or more genes for the pathway, enzyme, or protein of interest. In another embodiment, the microorganism is transformed to produce multiple copies of one or more genes for the pathway, enzyme, or protein of interest. In some embodiments, the polynucleotide transformed into the microorganism is heterologous. In other embodiments, the polynucleotide is derived from microorganism. In one embodiment, the microorganism is transformed with heterologous polynucleotides encoding one or more genes encoding enzymes for the fermentation of a hexose, wherein said genes are expressed at sufficient levels to confer upon said microorganism transformant the ability to produce ethanol at increased concentrations, productivity levels or yields compared to a microorganism that is not transformed. In another embodiment, the microorganism is transformed with heterologous polynucleotides encoding one or more genes encoding enzymes for the fermentation of a pentose, wherein said genes are expressed at sufficient levels to confer upon said microorganism transformant the ability to produce ethanol or other end-products at increased concentrations, productivity levels or yields compared to a microorganism that is not transformed. In still other embodiments, the microorganism is transformed with a combination of enzymes for fermentation of hexose and pentose saccharides. In some embodiments, an enhanced rate of end-product production can be achieved. In another embodiment, the microorganism is transformed with heterologous polynucleotides encoding one or more genes encoding saccharolytic enzymes for the saccharification of a polysaccharide, wherein said genes are expressed at sufficient levels to confer upon the transformed microorganism an ability to saccharify a polysaccharide to mono-, di- or oligosaccharides at increased concentrations, rates of saccharification or yields of mono-, di- or oligosaccharides compared to a microorganism that is not transformed.

In another embodiment the genetic modification is to a nucleic acid sequence a Clostridium phytofermentans. In one embodiment, the Clostridium phytofermentans is transformed with polynucleotides encoding one or more genes for the pathway, enzyme, or protein of interest. In another embodiment, the Clostridium phytofermentans is transformed to produce multiple copies of one or more genes for the pathway, enzyme, or protein of interest. In some embodiments, the polynucleotide transformed into the Clostridium phytofermentans is heterologous. In other embodiments, the polynucleotide is derived from Clostridium phytofermentans. In one embodiment, the Clostridium phytofermentans is transformed with heterologous polynucleotides encoding one or more genes encoding enzymes for the fermentation of a hexose, wherein said genes are expressed at sufficient levels to confer upon said Clostridium phytofermentans transformant the ability to produce ethanol at increased concentrations, productivity levels or yields compared to a Clostridium phytofermentans that is not transformed. In another embodiment, the Clostridium phytofermentans is transformed with heterologous polynucleotides encoding one or more genes encoding enzymes for the fermentation of a pentose, wherein said genes are expressed at sufficient levels to confer upon said Clostridium phytofermentans transformant the ability to produce ethanol or other end-products s at increased concentrations, productivity levels or yields compared to a Clostridium phytofermentans that is not transformed. In still other embodiments, the Clostridium phytofermentans is transformed with a combination of enzymes for fermentation of hexose and pentose saccharides. In some embodiments, an enhanced rate of end-product production can be achieved.

In another embodiment, the Clostridium phytofermentans is transformed with heterologous polynucleotides encoding one or more genes encoding saccharolytic enzymes for the saccharification of a polysaccharide, wherein said genes are expressed at sufficient levels to confer upon said Clostridium phytofermentans transformant the ability to saccharify a polysaccharide to mono-, di- or oligosaccharides at increased concentrations, rates of saccharification or yields of mono-, di- or oligosaccharides compared to a Clostridium phytofermentans that is not transformed. The production of a saccharolytic enzyme by the host, and the subsequent release of that saccharolytic enzyme into the medium, reduces the amount of commercial enzyme necessary to degrade biomass or polysaccharides into fermentable monosaccharides and oligosaccharides. The saccharolytic DNA can be native to the host, although more often the DNA will be foreign, i.e., heterologous. Advantageous saccharolytic genes include cellulolytic, xylanolytic, and starch-degrading enzymes such as cellulases, xylanases, glucanases, glucosidases, and amylases. The saccharolytic enzymes can be at least partially secreted by the host, or it can be accumulated substantially intracellularly for subsequent release. Advantageously, intracellularly-accumulated enzymes which are thermostable can be released when desired by heat-induced lysis. Combinations of enzymes can be encoded by the heterologous DNA, some of which are secreted, and some of which are accumulated.

In another embodiment further modifications can be made to enhance the end-product (e.g., ethanol) production by a recombinant microorganism. In one embodiment, a recombinant microorganism can further comprise an additional heterologous DNA segment, the expression product of which is a protein involved in the transport of mono- and/or oligosaccharides into the recombinant host. Likewise, additional genes from the glycolytic pathway can be incorporated into the host. In such ways, an enhanced rate of ethanol production can be achieved.

In order to improve the production of fermentation end-products (e.g. ethanol), modifications can be made in transcriptional regulators, genes for the formation of organic acids, carbohydrate transporter genes, sporulation genes, genes that influence the formation/regenerate of enzymatic cofactors, genes that influence ethanol tolerance, genes that influence salt tolerance, genes that influence growth rate, genes that influence oxygen tolerance, genes that influence catabolite repression, genes that influence hydrogen production, genes that influence resistance to heavy metals, genes that influence resistance to acids or genes that influence resistance to aldehydes.

Those skilled in the art will appreciate that a number of modifications can be made to the methods exemplified herein. For example, a variety of promoters can be utilized to drive expression of the heterologous genes in the recombinant Clostridium phytofermentans host. The skilled artisan, having the benefit of the instant disclosure, will be able to readily choose and utilize any one of the various promoters available for this purpose. Similarly, skilled artisans, as a matter of routine preference, can utilize a higher copy number plasmid. In another embodiment, constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target organism. This DNA can be ligated to form circles without replicons and used for transformation. Thus, the gene of interest can be introduced in a heterologous host such as E. coli, and short, random fragments can be isolated and ligated in Clostridium phytofermentans to promote homologous recombination.

In other embodiments, Clostridium phytofermentans isolates can be obtained without the use of recombinant DNA techniques that exhibit desirable properties such as increased productivity, increased yield, or increased titer. For example, mutagenesis, or random mutagenesis can be performed by chemical means or by irradiation of the microorganism. The population of mutagenized microorganisms can then be screened for beneficial mutations that exhibit one or more desirable properties. Screening can be performed by growing the mutagenized organisms on substrates that comprise carbon sources that will be utilized during the generation of end-products by fermentation. Screening can also include measuring the production of end-products during growth of the organism, or measuring the digestion or assimilation of the carbon source(s). The isolates so obtained can further be transformed with recombinant polynucleotides or used in combination with any of the methods and compositions provided herein to further enhance biofuel production.

### Biofuel Plant and Process of Producing Biofuel

In one aspect, provided herein is a fuel plant that includes a hydrolysis unit configured to hydrolyze a biomass material that includes a high molecular weight carbohydrate, and a fermentor configured to house a medium and contains microorganisms dispersed therein. In one embodiment the microorganism is Clostridium phytofermentans.

In another aspect, provided herein are methods of making a fuel or chemical end product that includes combining a microorganism (such as Clostridium phytofermentans cells or a similar C5/C6 Clostridium species) and a lignocellulosic material (and/or other biomass material) in a medium, and fermenting the lignocellulosic material under conditions and for a time sufficient to produce a fermentation end-product, such as a fuel (e.g., ethanol, propanol, methane or hydrogen).

In some embodiments, a process is provided for producing a fermentation end-product (such as ethanol or hydrogen) from biomass using acid hydrolysis pretreatment. In some embodiments, a process is provided for producing a fermentation end-product (such as ethanol or hydrogen) from biomass using enzymatic hydrolysis pretreatment. In another embodiment a process is provided for producing a fermentation end-product (such as ethanol or hydrogen) from biomass using biomass that has not been enzymatically pretreated. In another embodiment a process is provided for producing a fermentation end-product (such as ethanol or hydrogen) from biomass using biomass that has not been chemically or enzymatically pretreated, but is optionally steam treated.

In another aspect, provided herein are end-products made by any of the processes described herein.

Those skilled in the art will appreciate that a number of modifications can be made to the methods exemplified herein. For example, a variety of promoters can be utilized to drive expression of the heterologous genes in a recombinant microorganism (such as Clostridium phytofermentans). The skilled artisan, having the benefit of the instant disclosure, will be able to readily choose and utilize any one of the various promoters available for this purpose. Similarly, skilled artisans, as a matter of routine preference, can utilize a higher copy number plasmid. In another embodiment, constructs can be prepared for chromosomal integration of the desired genes. Chromosomal integration of foreign genes can offer several advantages over plasmid-based constructions, the latter having certain limitations for commercial processes. Ethanologenic genes have been integrated chromosomally in E. coli B; see Ohta et al. (1991) Appl. Environ. Microbiol. 57:893-900. In general, this is accomplished by purification of a DNA fragment containing (1) the desired genes upstream from an antibiotic resistance gene and (2) a fragment of homologous DNA from the target organism. This DNA can be ligated to form circles without replicons and used for transformation. Thus, the gene of interest can be introduced in a heterologous host such as E. coli, and short, random fragments can be isolated and ligated in Clostridium phytofermentans to promote homologous recombination.

Large Scale Fermentation End-Product Production from Biomass

In one aspect a fermentation end-product (e.g., ethanol) from biomass is produced on a large scale utilizing a microorganism, such as C. phytofermentans. In one embodiment, one first hydrolyzes a biomass material that includes high molecular weight carbohydrates to lower molecular weight carbohydrates, and then ferments the lower molecular weight carbohydrates utilizing of microbial cells to produce ethanol. In another embodiment, one ferments the biomass material itself without chemical and/or enzymatic pretreatment. In the first method, hydrolysis can be accomplished using acids, e.g., Bronsted acids (e.g., sulfuric or hydrochloric acid), bases, e.g., sodium hydroxide, hydrothermal processes, steam explosion, ammonia fiber explosion processes (“AFEX”), lime processes, enzymes, or combination of these. Hydrogen, and other products of the fermentation can be captured and purified if desired, or disposed of, e.g., by burning. For example, the hydrogen gas can be flared, or used as an energy source in the process, e.g., to drive a steam boiler, e.g., by burning. Hydrolysis and/or steam treatment of the biomass can, e.g., increase porosity and/or surface area of the biomass, often leaving the cellulosic materials more exposed to the microbial cells, which can increase fermentation rate and yield. Removal of lignin can, e.g., provide a combustible fuel for driving a boiler, and can also, e.g., increase porosity and/or surface area of the biomass, often increasing fermentation rate and yield. In some embodiments, the initial concentration of the carbohydrates in the medium is greater than 20 mM, e.g., greater than 30 mM, 50 mM, 75 mM, 100 mM, 150 mM, 200 mM, or even greater than 500 mM.

Biomass Processing Plant and Process of Producing Products from Biomass

In one aspect, the invention features a fuel plant that includes a hydrolysis unit configured to hydrolyze a biomass material that includes a high molecular weight carbohydrate, a fermentor configured to house a medium with a C5/C6 hydrolyzing microorganism (e.g., Clostridium phytofermentans) dispersed therein, and one or more product recovery system(s) to isolate an end-product or end-products and associated by-products and co-products.

In another aspect, the invention features methods of making an end-product or end-products that include combining a C5/C6 hydrolyzing microorganism (e.g., Clostridium phytofermentans) and a biomass feed in a medium, and fermenting the biomass material under conditions and for a time sufficient to produce a biofuel, chemical product or fermentation end-products (e.g. ethanol, propanol, hydrogen, lignin, terpenoids, and the like).

In another aspect, the invention features end-products made by any of the processes described herein.

Large Scale Production of Fermentation End-Products from Biomass

Generally, there are two basic approaches to producing one or more fermentation end-products from biomass on a large scale utilizing a C5/C6 hydrolyzing microorganism (e.g., Clostridium phytofermentans). In all methods, depending on the type of biomass and its physical manifestation, one of the processes can comprise a milling of the carbonaceous material, via wet or dry milling, to reduce the material in size and increase the surface to volume ratio (physical modification).

In one embodiment, a biomass material comprising includes high molecular weight carbohydrates is hydrolyzed to delignify it or to separate the carbohydrate compounds from noncarbohydrate compounds. Using a combination of heat, chemical, and/or enzymatic treatment, the hydrolyzed material can be separated to form liquid and dewatered streams, which can be separately treated and kept separate or recombined, and then ferments the lower molecular weight carbohydrates utilizing a C5/C6 hydrolyzing microorganism (e.g., Clostridium phytofermentans) to produce one or more chemical products. In the second method, one ferments the biomass material itself without heat, chemical, and/or enzymatic pretreatment. In the first method, hydrolysis can be accomplished using acids (e.g. sulfuric or hydrochloric acids), bases (e.g. sodium hydroxide), hydrothermal processes, ammonia fiber explosion processes (“AFEX”), lime processes, enzymes, or combination of these. Hydrolysis and/or steam treatment of the biomass can, e.g., increase porosity and/or surface area of the biomass, often leaving the cellulosic materials more exposed to a C5/C6 hydrolyzing microorganism (e.g., Clostridium phytofermentans), which can increase fermentation rate and yield. Hydrolysis and/or steam treatment of the biomass can, e.g., produce by-products or co-products which can be separated or treated to improve fermentation rate and yield, or used to produce power to run the process, or used as products with or without further processing. Removal of lignin can, e.g., provide a combustible fuel for driving a boiler. Gaseous (e.g., methane, hydrogen or CO₂), liquid (e.g. ethanol and organic acids), or solid (e.g. lignin), products of the fermentation can be captured and purified if desired, or disposed of, e.g., by burning. For example, the hydrogen gas can be flared, or used as an energy source in the process, e.g., to drive a steam boiler, e.g., by burning. Products exiting the fermentor can be further processed, e.g. ethanol can be transferred to distillation and rectification, producing a concentrated ethanol mixture or solids can be separated for use to provide energy or as chemical products.

In some embodiments, the treatment includes a step of treatment with acid. In some embodiments, the acid is dilute. In some embodiments, the acid treatment is carried out at elevated temperatures of between about 85 and 140° C. In some embodiments, the method further comprises the recovery of the acid treated biomass solids, for example by use of a sieve. In some embodiments, the sieve comprises openings of approximately 150-250 microns in diameter. In some embodiments, the method further comprises washing the acid treated biomass with water or other solvents. In some embodiments, the method further comprises neutralizing the acid with alkali. In some embodiments, the method further comprises drying the acid treated biomass. In some embodiments, the drying step is carried out at elevated temperatures between about 15-45° C. In some embodiments, the liquid portion of the separated material is further treated to remove toxic materials. In some embodiments, the liquid portion is separated from the solid and then fermented separately. In some embodiments, a slurry of solids and liquids are formed from acid treatment and then fermented together.

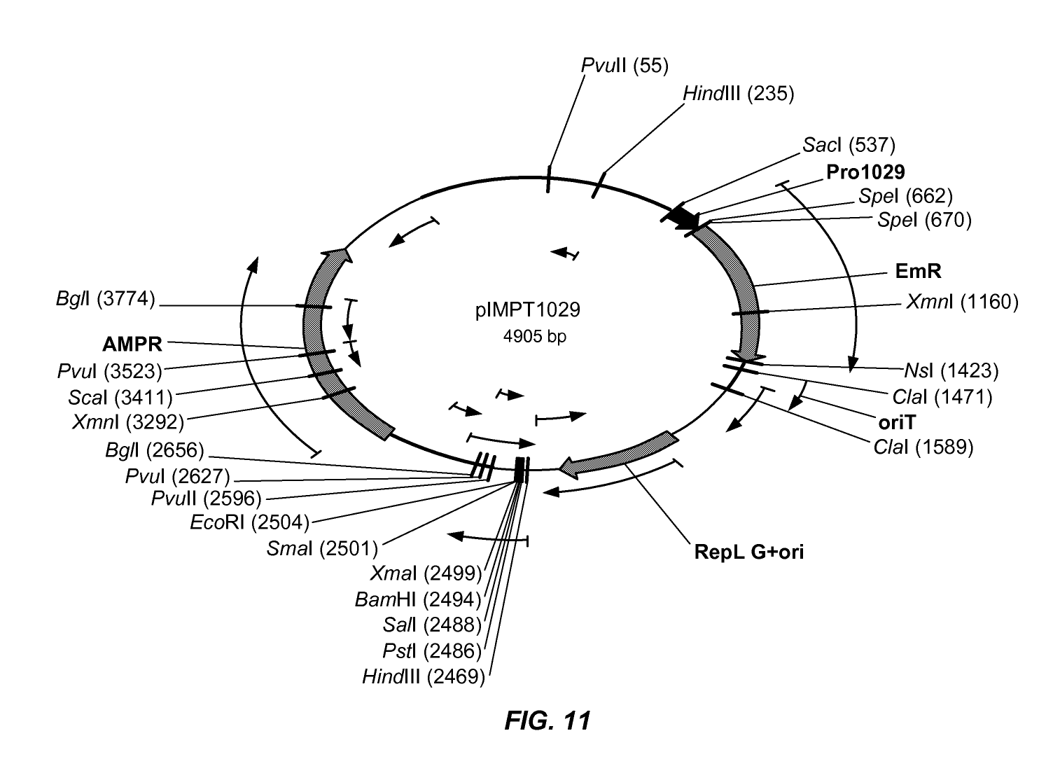

FIG. 11 illustrates an example of a method for producing chemical products from biomass by first treating biomass with an acid at elevated temperature and pressure in a hydrolysis unit. The biomass can first be heated by addition of hot water or steam. The biomass can be acidified by bubbling gaseous sulfur dioxide through the biomass that is suspended in water, or by adding a strong acid, e.g., sulfuric, hydrochloric, or nitric acid with or without preheating/presteaming/water addition. During the acidification, the pH is maintained at a low level, e.g., below about 5. The temperature and pressure can be elevated after acid addition. In addition to the acid already in the acidification unit, optionally, a metal salt such as ferrous sulfate, ferric sulfate, ferric chloride, aluminum sulfate, aluminum chloride, magnesium sulfate, or mixtures of these can be added to aid in the hydrolysis of the biomass. The acid-impregnated biomass is fed into the hydrolysis section of the pretreatment unit. Steam is injected into the hydrolysis portion of the pretreatment unit to directly contact and heat the biomass to the desired temperature. The temperature of the biomass after steam addition is, e.g., between about 130° C. and 220° C. The hydrolysate is then discharged into the flash tank portion of the pretreatment unit, and is held in the tank for a period of time to further hydrolyze the biomass, e.g., into oligosaccharides and monomeric sugars. Steam explosion can also be used to further break down biomass. Alternatively, the biomass can be subject to discharge through a pressure lock for any high-pressure pretreatment process. Hydrolysate is then discharged from the pretreatment reactor, with or without the addition of water, e.g., at solids concentrations between about 15% and 60%.

After pretreatment, the biomass can be dewatered and/or washed with a quantity of water, e.g. by squeezing or by centrifugation, or by filtration using, e.g. a countercurrent extractor, wash press, filter press, pressure filter, a screw conveyor extractor, or a vacuum belt extractor to remove acidified fluid. The acidified fluid, with or without further treatment, e.g. addition of alkali (e.g. lime) and or ammonia (e.g. ammonium phosphate), can be re-used, e.g., in the acidification portion of the pretreatment unit, or added to the fermentation, or collected for other use/treatment. Products can be derived from treatment of the acidified fluid, e.g., gypsum or ammonium phosphate. Enzymes or a mixture of enzymes can be added during pretreatment to assist, e.g. endoglucanases, exoglucanases, cellobiohydrolases (CBH), beta-glucosidases, glycoside hydrolases, glycosyltransferases, lyases, and esterases active against components of cellulose, hemicelluloses, pectin, and starch, in the hydrolysis of high molecular weight components.

The fermentor is fed with hydrolyzed biomass, any liquid fraction from biomass pretreatment, an active seed culture of Clostridium phytofermentans cells, if desired a co-fermenting microbe, e.g., yeast or E. coli, and, if required, nutrients to promote growth of Clostridium phytofermentans or other microbes. Alternatively, the pretreated biomass or liquid fraction can be split into multiple fermentors, each containing a different strain of Clostridium phytofermentans and/or other microbes, and each operating under specific physical conditions. Fermentation is allowed to proceed for a period of time, e.g., between about 15 and 150 hours, while maintaining a temperature of, e.g., between about 25° C. and 50° C. Gas produced during the fermentation is swept from fermentor and is discharged, collected, or flared with or without additional processing, e.g. hydrogen gas can be collected and used as a power source or purified as a co-product.

After fermentation, the contents of the fermentor are transferred to product recovery. Products are extracted, e.g., ethanol is recovered through distilled and rectification.

Chemical Production from Biomass without Pretreatment

FIG. 12 depicts a method for producing chemicals from biomass by charging biomass to a fermentation vessel. The biomass can be allowed to soak for a period of time, with or without addition of heat, water, enzymes, or acid/alkali. The pressure in the processing vessel can be maintained at or above atmospheric pressure. Acid or alkali can be added at the end of the pretreatment period for neutralization. At the end of the pretreatment period, or at the same time as pretreatment begins, an active seed culture of a C5/C6 hydrolyzing microorganism (e.g., Clostridium phytofermentans) and, if desired, a co-fermenting microbe, e.g., yeast or E. coli, and, if required, nutrients to promote growth of a C5/C6 hydrolyzing microorganism (e.g., Clostridium phytofermentans) are added. Fermentation is allowed to proceed as described above. After fermentation, the contents of the fermentor are transferred to product recovery as described above.

Any combination of the chemical production methods and/or features can be utilized to make a hybrid production method. In any of the methods described herein, products can be removed, added, or combined at any step. A C5/C6 hydrolyzing microorganism (e.g., Clostridium phytofermentans) can be used alone or synergistically in combination with one or more other microbes (e.g. yeasts, fungi, or other bacteria). In some embodiments different methods can be used within a single plant to produce different end-products.

In another aspect, the invention features a fuel plant that includes a hydrolysis unit configured to hydrolyze a biomass material that includes a high molecular weight carbohydrate, and a fermentor configured to house a medium and contains a C5/C6 hydrolyzing microorganism (e.g., Clostridium phytofermentans) dispersed therein.

In another aspect, the invention features methods of making a fuel or fuels that include combining a C5/C6 hydrolyzing microorganism (e.g., Clostridium phytofermentans) and a lignocellulosic material (and/or other biomass material) in a medium, and fermenting the lignocellulosic material under conditions and for a time sufficient to produce a fuel or fuels, e.g., ethanol, propanol and/or hydrogen or another chemical compound.

In some embodiments, the present invention provides a process for producing ethanol and hydrogen from biomass using acid hydrolysis pretreatment. In some embodiments, the present invention provides a process for producing ethanol and hydrogen from biomass using enzymatic hydrolysis pretreatment. Other embodiments provide a process for producing ethanol and hydrogen from biomass using biomass that has not been enzymatically pretreated. Still other embodiments disclose a process for producing ethanol and hydrogen from biomass using biomass that has not been chemically or enzymatically pretreated, but is optionally steam treated.

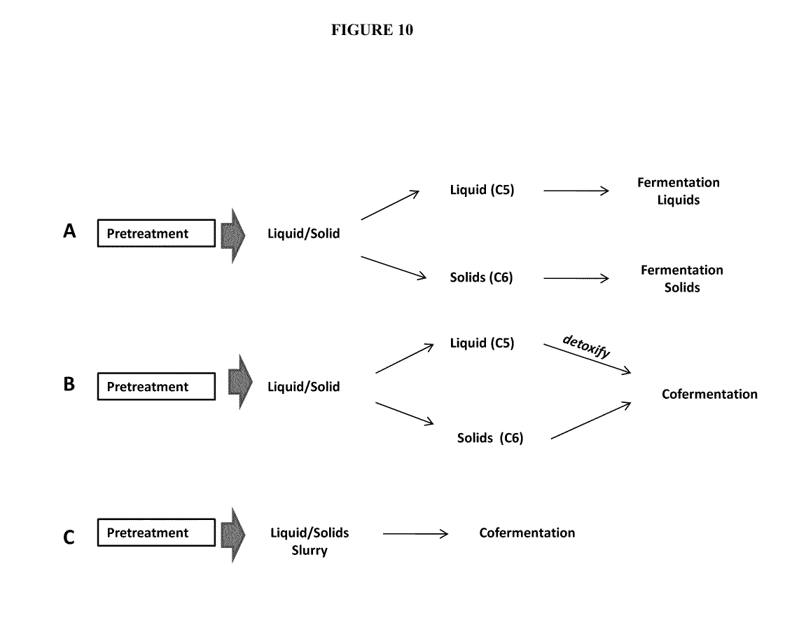

FIG. 10 discloses pretreatments that produce hexose or pentose saccharides or oligomers that are then unprocessed or processed further and either, fermented separately or together. FIG. 10A depicts a process (e.g., acid pretreatment) that produces a solids phase and a liquid phase which are then fermented separately. FIG. 10B depicts a similar pretreatment that produces a solids phase and liquids phase. The liquids phase is separated from the solids and elements that are toxic to the fermenting microorganism are removed prior to fermentation. At initiation of fermentation, the two phases are recombined and cofermented together. This is a more cost-effective process than fermenting the phases separately. The third process (FIG. 10C is the least costly. The pretreatment results in a slurry of liquids or solids that are then cofermented. There is little loss of saccharides component and minimal equipment required.

### EXAMPLES

### Example 1

### Biomass Processing and Pretreatment Procedure

Corn stalks were chopped in a knife mill to ¼^{th} inch sizes followed by screening through 2 mm sieve. Screened corn stover was mixed with water to prepare a 10% (w/v) slurry. Alkaline digestion of the corn stover slurry was performed in an autoclave at 121° C. and 15 PSI for 2 hours using 0.2 g NaOH per g corn stover.

Digested corn stover was washed with tap water (5-7 volumes) to neutralize pH. Following neutralization solids were recovered by filtration through a 250 micron sieve and dried at 35 deg C. to the moisture content of about 5%. Dried clumps were ground prior to adding to the fermentor.

Degassing and Sterilization Procedure of the Media:

All serum vials used for inoculums propagation were degassed under vacuum (about 400 mbar absolute pressure), for about 5 minutes, at room temperature), with the vacuum broken under a nitrogen purge. A minimum of three degassing cycles were performed. The serum vials, media and fermentor vessel were sterilized by autoclaving at 121° C. temperature and 15 PSI pressure for 30 minutes. The fermentor was sparged with N₂ gas for over an hour to lower the redox potential to around −300 mV prior to inoculation.

Inoculum Preparation:

Frozen culture of Clostridium phytofermentans was propagated at 35° C. for 48 hours in 10 mL tubes containing 0.3% cellobiose along with 1.5 g/L KH₂PO₄, 2.9 g/L K₂HPO₄, 4.6 g/L ammonium sulphate, 2 g/L cysteine-HCl, 1 g/L MgCl₂ 6H₂O, 0.15 g/L CaCl₂ 2H₂O, 0.00125 g/L FeSO₄ 7H₂O in DI water. The pH of the media was adjusted to 7.5 with 2 N NaOH. Following propagation in test tube the inoculums were grown at 35° C. for 24 hours in 100 mL serum vials using 2% (v/v) seed size. The serum vials had 20 g/L malt syrup, 1.5 g/L KH₂PO₄, 2.9 g/L K₂HPO₄, 4.6 g/L ammonium sulfate, 2 g/L cysteine-HCl, 3 g/L sodium citrate, 1 g/L MgCl₂ 6H₂O, 0.15 g/L CaCl₂ 2H₂O, 0.00125 g/L FeSO₄ 7H₂O in DI water.

Simultaneous Hydrolysis and Fermentation of Biomass:

A 5 L stirred tank reactor was operated at 2 L starting volume under fed-batch mode. The media contained 50 g/L of pretreated corn stover along with 3 g/L K₂HPO₄, 1.6 g/L KH₂PO₄, 2 g/L TriSodium citrate.2H₂O, 1.2 g/L citric acid H₂O, 0.5 g/L (NH₄)₂SO₄, 1 g/L NaCl, 0.8 g/L MgCl₂.6H₂O, 0.1 g/L CaCl₂.2H₂O, 0.00125 g/L FeSO₄.7H₂O, 1 g/L CysteineHCl, 10 g/L yeast extract (Bacto), along with 5 g/L of corn steep powder dissolved in DI water.

In some instances 50 mL of CelluSeb-TL was added to the fermentor through 0.2 micron filter prior to inoculation to enhance hydrolysis and increase yield. Then the fermentor was sparged with N₂ gas for over an hour to lower the redox potential to around −300 mV followed by inoculation with 20 ml of the concentrated inoculums. The operating pH and temperature was 6.5 and 35° C., respectively, and the fermentor was continuously agitated at 300 rpm.

A bolus of 25-50 g of pretreated corn stover was given at regular time intervals. Additional doses of 7.5 and 25 mL enzyme were given at 72 and 240 hours post inoculation.

Sample Collection and Analysis:

Samples were collected at time intervals and analyzed for sugars, organic acids and ethanol using HPLC equipped with Aminex® HPX-87H Exclusion column (300 mm×7.8 mm) and RI detector. 0.01 N H₂SO₄ was used as the mobile phase at 0.6 mL/minute, and the column was maintained at 55° C.

Results

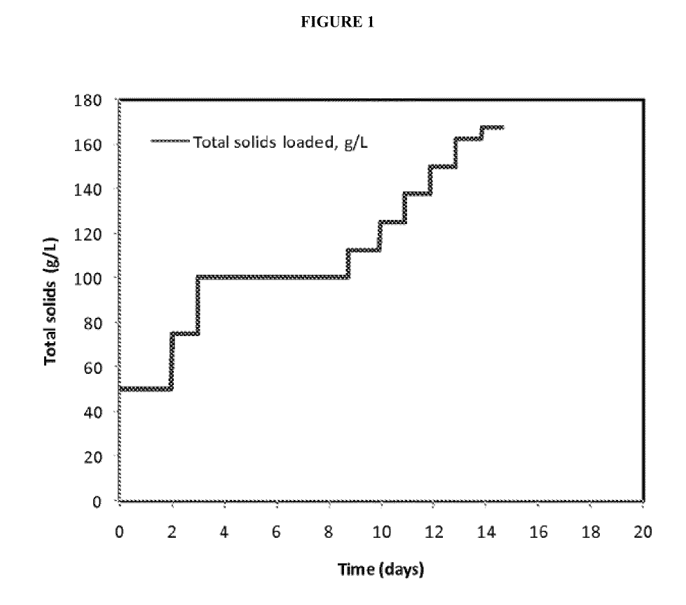

The fermentation was initiated with a solids loading of 50 g/L. Additional bolus feeds of 25 g/L pretreated corn stover solids were given on 3^{rd} and 4^{th} day. Initial ethanol production rate was observed to be about 10 g/L-d, which slowed down to about 2 g/L-d in between 4^{th} to 8^{th} day without further addition of corn stover. From the 9^{th} day onward solids bolus feed quantity was reduced to 12.5 g/L and was administered every 24 hours as shown in FIG. 1. This helped in improving the ethanol production rate to above 3 g/L-d.

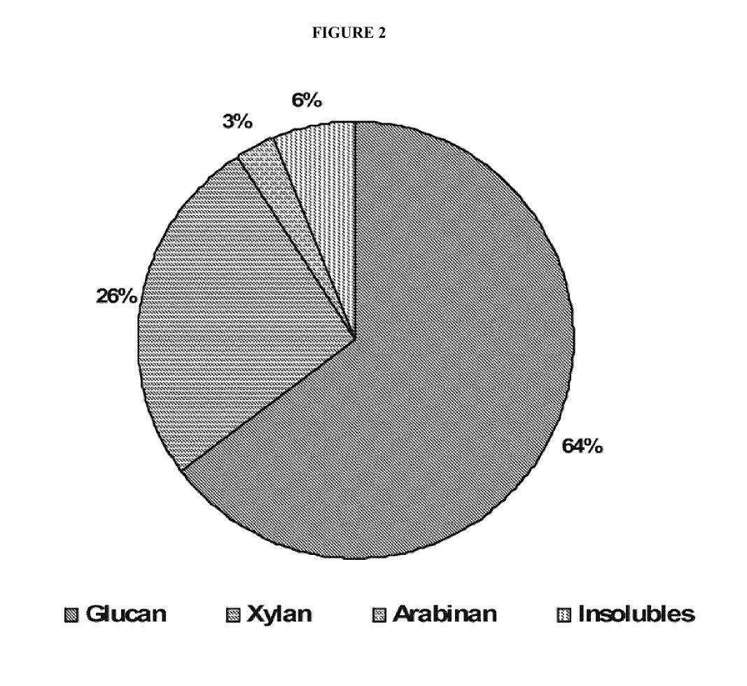

Ethanol was major product of the fermentation. Compositional analysis of the pretreated corn stover was performed using acid hydrolysis. The results of the compositional analysis showed the percentage of glucan, xylan, arabinan and insolubles as 64%, 26%, 3% and 6%, respectively (FIG. 2). Based on the composition the amount of fermentables in the pretreated corn stover was about 93%. Assuming a saccharification efficiency of 90%, the observed ethanol yield was calculated as 0.39 g per g of biomass loaded.

### Example 2

### Ethanol Production from Hexose and Pentose Saccharides

Batch fermentation was performed to produce ethanol through simultaneous fermentation of hexose (glucose, cellobiose) and pentose (xylose and arabinose) sugars using Clostridium phytofermentans in stirred tank reactors.

### Chemicals Used:

All chemical used in this experiment were of reagent grade from Sigma-Aldrich. Degassing and sterilization procedure:

All reactors and serum vials used for inoculum propagation were degassed under vacuum (about 400 mbar absolute pressure), for about 5 minutes, at room temperature), with the vacuum broken under a nitrogen purge. A minimum of three degassing cycles were performed. The vessel was sterilized by autoclaving at 121° C. temperature and 15 PSI pressure for 30 minutes.

### Inoculum Preparation:

A frozen culture of Clostridium phytofermentans was cultured and expanded at 35° C. for 48 hours in 10 mL tubes containing 0.3% cellobiose along with 1.5 g/L KH2PO4, 2.9 g/L K2HPO4, 4.6 g/L ammonium sulfate, 2 g/L cysteine-HCl, 1 g/L MgCl2 6H2O, 0.15 g/L CaCl2 2H2O, 0.00125 g/L FeSO4 7H2O in DI water. The pH of the media was adjusted to 7.5 with 2 N NaOH. After autoclaving, the inoculum was grown at 35° C. for 24 hours in 100 mL serum using 2% (v/v) seed size. The serum vials contained 20 g/L cellobiose, 1.5 g/L KH2PO4, 2.9 g/L K2HPO4, 4.6 g/L ammonium sulfate, 2 g/L cysteine-HCl, 3 g/L sodium citrate, 1 g/L MgCl2 6H2O, 0.15 g/L CaCl2 2H2O, 0.00125 g/L FeSO4 7H2O in DI water. Expanded inoculum was examined for purity and centrifuged at 3000 rpm for 15 minutes to generate 10 mL of concentrated biomass (2-4 g/L total suspended solids) to be used as inoculum for each fermentor.

### Simultaneous Fermentation of Hexose and Pentose Sugars:

Two stir tank reactors of 400 mL working volume were operated under batch mode. In bioreactor, BR1, 30 g/L of cellobiose and 30 g/L of xylose were used as carbon source along with 3 g/L K2HPO4, 1.6 g/L KH2PO4, 2 g/L TriSodium citrate.2H2O, 1.2 g/L citric acid H2O, 0.5 g/L (NH4)2SO4, 1 g/L NaCl, 0.8 g/L MgCl2.6H2O, 0.1 g/L CaCl2.2H2O, 0.00125 g/L FeSO4.7H2O, 1 g/L Cysteine HCl, 10 g/L yeast extract (Bacto), along with 5 g/L of corn steep powder dissolved in deionized water. The second reactor, BR2, contained 30 g/L of glucose and 30 g/L of xylose as carbon source along with the same nutrients as BR1. Each of the fermentors was inoculated with 10 ml of the inoculum. The fermentors were operated at 35° C. and pH 6.5 and continuously mixed at 300 rpm.

Samples were collected at different time intervals and analyzed for sugars, organic acids and ethanol using HPLC equipped with Aminex® HPX-87H Exclusion column (300 mm×7.8 mm) and RI detector. 0.005 NH2SO4 was used as the mobile phase at 0.6 mL/minute, and the column was maintained at 55° C.

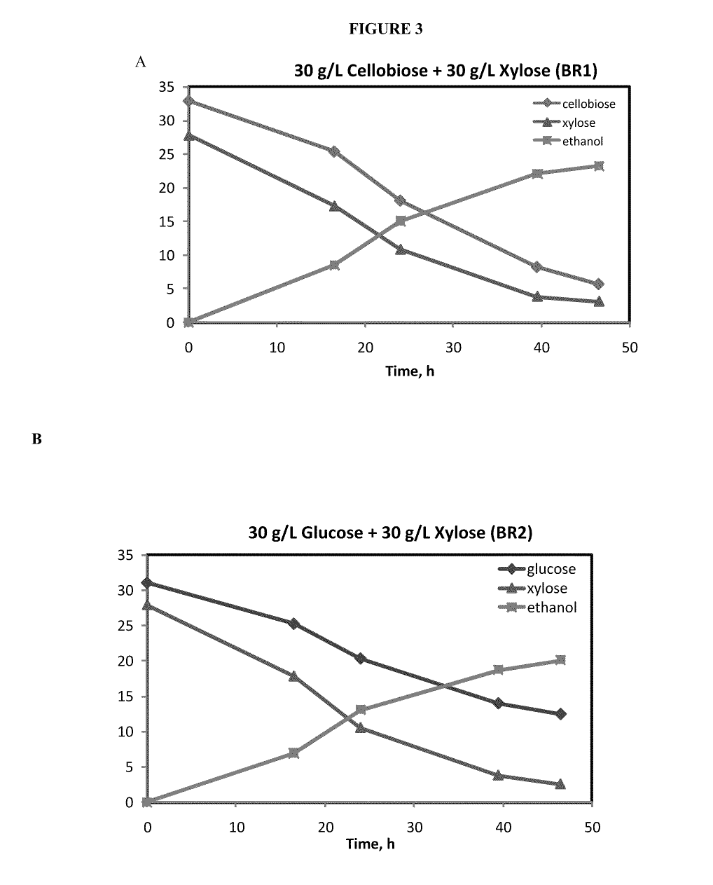

The results as shown in Table 1 and FIGS. 3A and 3B depict the robust utilization of both hexose and pentose saccharide substrates and conversion of those substrates into ethanol during the fermentation process. The results also suggest that pentose saccharides (e.g. xylose) are converted to ethanol at least as rapidly and completely as hexose saccharides (e.g. cellobiose, or glucose).

TABLE 1
Concentration of sugars and ethanol

| Run | BR1 | | | BR2 | | |
|---|---|---|---|---|---|---|
| time | Cellobiose | Xylose | Ethanol | Glucose | Xylose | Ethanol |
| (h) | (g/L) | (g/L) | (g/L) | (g/L) | (g/L) | (g/L) |
| 0 | 32.93 | 27.8 | 0 | 31.06 | 27.92 | 0 |
| 16.5 | 25.41 | 17.29 | 8.46 | 25.26 | 17.8 | 6.94 |
| 24 | 18.12 | 10.84 | 15.07 | 20.35 | 10.52 | 13.1 |
| 39.5 | 8.24 | 3.85 | 22.07 | 14 | 3.78 | 18.77 |
| 46.5 | 5.7 | 3.11 | 23.22 | 12.48 | 2.53 | 20.11 |

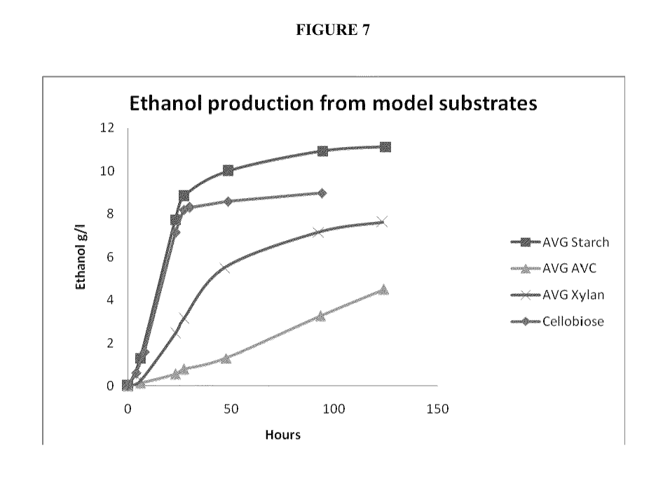

The normalized carbohydrate utilization results of the BR1 and BR2 fermentation runs are depicted in FIG. 7 which allows a more direct comparison between the different carbon sources. FIG. 7 further suggests that pentose carbohydrates (e.g. xylose) are converted to ethanol at least as well as hexose carbohydrates (e.g. cellobiose, or glucose).

### Example 3

### Ethanol Production from Starch, Cellobiose, and Xylose

Batch fermentation was performed to produce ethanol by simultaneous fermentation of hexose (starch, cellobiose) and pentose (xylose) sugars using Clostridium phytofermentans.

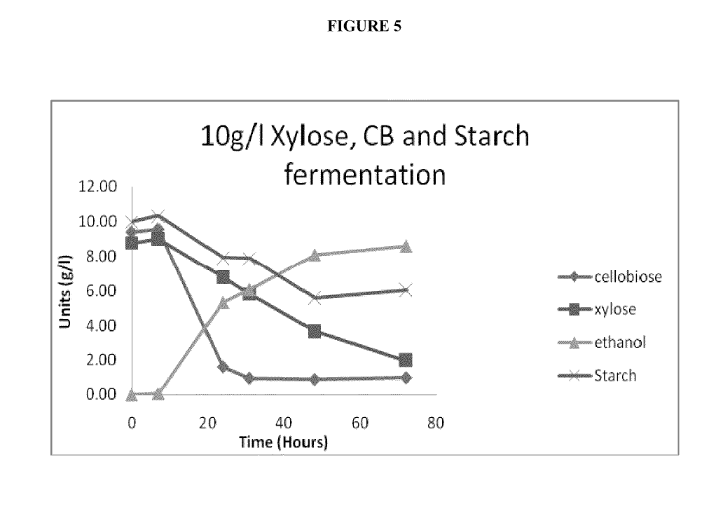

A 10 g/L mixture of alpha-1,4-linked glucan (starch), beta-1,4-linked glucan (cellobiose), and xylose was incubated with a Clostridium phytofermentans derived strain at 35° C. for 48 hours. The fermentation results shown in FIG. 5 and in Table 2 below indicate that the organism is capable of simultaneous usage and conversion to ethanol of all three carbon sources.

TABLE 2
Concentration of sugars and ethanol

| | T₀, | T₁, | T₂, | T₃, | T₄, |
|---|---|---|---|---|---|
| | Jan. 13, 2009 | Jan. 13, 2010 | Jan. 14, 2010 | Jan. 14, 2010 | Jan. 15, 2010 |
| | 0 hrs | 7 hrs | 24 hrs | 31 hrs | 48 hrs |
| ethanol produced (g/l) | 0.0 | 0.1 | 5.3 | 6.1 | 8.1 |
| consumed cellobiose (g/l) | 0.0 | 0.2 | 7.8 | 8.4 | 8.5 |
| consumed xylose (g/l) | 0.0 | 0.2 | 2.0 | 3.0 | 5.1 |
| calculated consumed starch (g/l) | 0.0 | −0.3 | 2.1 | 2.1 | 4.4 |
| Assumed yield (g/l) | 0.45 | 0.45 | 0.45 | 0.45 | 0.45 |

### Example 4

### Ethanol Production from Hexose (Glucose) and Pentose (Xylose, Arabinose)

Batch fermentation was performed to produce ethanol by fermentation of hexose (glucose) or pentose (xylose, arabinose) using Clostridium phytofermentans.

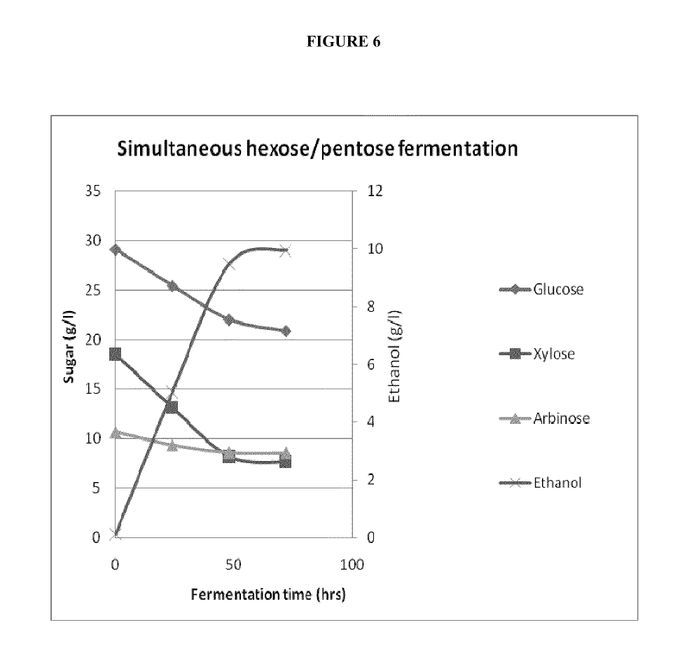

Batch fermentation reactions were set up to test ethanol production from various carbohydrate substrates. Fermentation medium comprising a mixture of xylose, glucose, and arabinose was prepared. The resulting medium was then incubated with a Clostridium phytofermentans derived strain at 35° C. for 48 hours. The fermentation results shown in FIG. 6 indicate that the organism is capable of efficient and rapid utilization of all three carbon sources, and that the organism is able to produce at least about 25-30 g/L of ethanol in about 48 hours.

### Example 5

### Ethanol Production from Starch, Microcrystalline Cellulose, Xylan, and Cellobiose

Batch fermentation was performed to produce ethanol by fermentation of hexose (starch, microcrystalline cellulose, cellobiose) or pentose (xylan) sugars using Clostridium phytofermentans.

Batch fermentation reactions were set up to test ethanol production from various carbohydrate substrates. Four different fermentation media were tested comprising: 1) 30 g/L xylan, 2) 30 g/L starch, 3) 30 g/L Avicel microcrystalline cellulose (AVC), and 20 g/L cellobiose. The resulting media were then incubated with a Clostridium phytofermentans derived strain at 35° C. for 48 hours. The fermentation results shown in FIG. 7 and in Table 3 below indicate that the organism is capable of efficient and rapid conversion to ethanol of all four carbon sources, with conversion of microcrystalline cellulose exhibiting the slowest ethanol productivity, followed by xylan, cellobiose, and starch.

TABLE 3
Rates for substrate consumption through 48 hrs

| | | | | maximum | yield |
|---|---|---|---|---|---|
| | substrate | 48 hrs | 120 hrs | rate | g EtOH/g |
| | g/l initial | g/l ethanol | g/l ethanol | g/l/h | Substrate |
| Xylan | 30 | 5.5 | 7.61 | 0.15 | 0.25 |
| Starch | 30 | 9.99 | 11.12 | 0.35 | 0.37 |
| Avicel | 30 | 1.29 | 4.49 | 0.03 | 0.15 |
| Cellobiose | 20 | 8.68 | 8.96 | 0.33 | 0.45 |

### Example 6

### Genetic Modification of Clostridium phytofermentans to Produce Increased Biofuels, Including Ethanol

Plasmids suitable for use in C. phytofermentans were constructed using portions of plasmids obtained from bacterial culture collections. Plasmid pIMP1 is a non-conjugal plasmid that can replicate in E. coli as well as a range of gram positive bacterial species and it encodes for resistance to erythromycin. Wilde-type C. phytofermentans is highly sensitive to erythromycin. Wilde-type C. phytofermentans does not grow at concentrations of 0.5 micrograms of erythromycin per ml of microbial growth media. The broad host range conjugal plasmid RK2 contains all of the genes needed for a bacterial conjugation system which include: an origin of replication specific to the DNA polymerase of the conjugation system, conjugal DNA replication genes, and genes encoding for the synthesis of pili to enable the recognition of potential recipient bacterial cells and to serve as the conduit through which single stranded plasmid DNA is transferred by cell-to-cell contact from donor to recipient cells. The origin of transfer for the RK2 conjugal system was obtained from plasmid pRK29O which was obtained from the German Collection of Microorganisms and Cell Cultures (DSMZ) as DSM 3928, and the other conjugation functions of RK2 were obtained from pRK2013 which was obtained from DSMZ as DSM 5599. Polymerase chain reaction was used to amplify the 112 basepair origin of transfer region (oriT) from pRK29O using primers that added Cla1 restriction sites flanking the oriT region. This DNA fragment was inserted into the Cla1 site of pIMP1 to yield plasmid pIMPT. Polymerase chain reaction was used to amplify the promoter of the alcohol dehydrogenase gene C. phytofermentans 1029 from the C. phytofermentans chromosome and it was used to replace the promoter of the erythromycin gene in pIMPT to create pIMPT1029. When pRK2013 was also present to supply other conjugation functions pIMPT1029 could be conjugally transferred from E. coli to C. phytofermentans. Successful transfer of plasmid DNA into C. phytofermentans was demonstrated by virtue of the ability of the C. phytofermentans derivative containing pIMPT1029 to grow on media containing up to 10 micrograms per ml erythromycin and by use of PCR primers to specifically amplify two genetic regions specific to pIMPT1029 from the C. phytofermentans derivative but not from a control C. phytofermentans culture that did not contain the plasmid.

The method of accomplishing conjugal transfer of pIMPT1029 from E. coli to C. phytofermentans consisted of first constructing an E. coli strain (DH5alpha) that contains both pIMPT1029 and pRK2013. Then fresh cells of this E. coli culture and fresh cells of the C. phytofermentans recipient culture were obtained. The two bacterial cultures were then centrifuged to yield cell pellets and the pellets were resuspended in the same media to obtain cell suspensions that were concentrated about ten-fold and had cell densities of about 10¹⁰ cells per ml. These concentrated cell suspensions were then mixed to achieve a donor-to-recipient ratio of five-to-one. This cell suspension was spotted onto QM1 agar plates and incubated anaerobically at 30 degrees Centigrade for 24 hours. Then the cell mixture was removed from the QM1 plate and placed on solid or in liquid QM1 media containing antibiotics that selected for C. phytofermentans recipient cells that expressed erythromycin resistance. This was accomplished by using a combination of antibiotics that consisted of trimethoprim at 20 micrograms per ml, cycloserine at 250 micrograms per ml, and erythromycin at 10 micrograms per ml. The E. coli donor was unable to survive exposure to these concentrations of trimethoprim and cycloserine, while the wild-type C. phytofermentans recipient was unable to survive exposure to this concentration of erythromycin (but could tolerate trimethoprim and cycloserine at these concentrations). Accordingly, after incubation of these antibiotic-containing plates or liquid media for 5-to-7 days at 30 degrees Centigrade under anaerobic conditions derivates of C. phytofermentans were obtained that were erythromycin resistant and these C. phytofermentans derivatives were subsequently shown to contain pIMPT1029 as demonstrated by PCR analyses.

The surprising result was that the only a specially constructed derivative of the erythromycin resistance gene that contained the C. phytofermentans promoter from the alcohol dehydrogenase gene could be functionally expressed in C. phytofermentans.

Other genes of interest, either from C. phytofermentans or from heterologous sources will be introduced into the pIMPT construct and will be used to transform C. phytofermentans. Genes that will be used to transform C. phytofermentans include those that express gene products that increase the environmental tolerance of C. phytofermentans to ethanol, acidic pH, or other toxic intermediates encountered during the production of biofuels.

A map of the plasmid pIMPT1029 is produced in FIG. 11, along with the DNA sequence of this plasmid, provided as SEQ ID NO:1.

SEQ ID NO: 1:
gcgcccaatacgcaaaccgcctaccccgcgcgttggccgattcattaa
tgcagaggcacgacaggtttcccgactggaaagcgggcagtgagcgca
acgcaattaatgtgagttagacactcattaggcaccccaggctttaca
ctttatgatccggctcgtatgttgtgtggaattgtgagcggataacaa
tttcacacaggaaacagctatgaccatgattacgccaaagattggcta
acacacacgccattccaaccaatagttttctcggcataaagccatgac
tgacgataaatgcactaatgccttaaaaaaacattaaagtctaacaca
ctagacttatttacttcgtaattaagtcgttaaaccgtgtgactacga
ccaaaagtataaaacctttaagaactttatttttatgtaaaaaaagaa
actagataaatctacatatatttattcaataatcgcatcagattgcag
tataaatttaacgatcactcatcatgttcatatttatcagagacctta
tattttatttcgatttatttgttatttatttaacatttttctattgac
ctcatcttttctatgtgttattcttttgttaattgtttacaaataatc
tacgatacatagaaggaggaaaaactagtatactagtatgaacgagaa
aaatataaaacacagtcaaaactttattacttcaaaacataatataga
taaaataatgacaaatataagattaaatgaacatgataatatattgaa
atcggctcaggaaaagggcattttaccatgaattagtacagaggtgta
atttcgtaactgccattgaaatagaccataaattatgcaaaactacag
aaaataaacttgttgatcacgataatttccaagttttaaacaaggata
tattgcagtttaaatttcctaaaaaccaatcctataaaatatttggta
atataccttataacataagtacggatataatacgcaaaattgtttttg
atagtatagctgatgagatttatttaatcgtggaatacgggtttgcta
aaagattattaaatacaaaacgctcattggcattatttttaatggcag
aagttgatatttctatattaagtatggttccaagagaatattttcatc
ctaaacctaaagtgaatagacacttatcagattaaatagaaaaaaatc
aagaatatcacacaaagataaacagaagtataattatttcgttatgaa
atgggttaacaaagaatacaagaaaatatttacaaaaaatcaatttaa
caattccttaaaacatgcaggaattgacgatttaaacaatattagatt
gaacaattcttatctcttttcaatagctataaattatttaataagtaa
gttaagggatgcataaactgcatcccttaacttgtttttcgtgtacct
attttttgtgaatcgatccggccagcctcgcagagcaggattcccgtt
gagcaccgccaggtgcgaataagggacagtgaagaaggaacacccgct
cgcgggtgggcctacttcacctatcctgcccggatcgattatgtattt
gcgcattcacttatttctatataaatatgagcgaagcgaataagcgtc
ggaaaagcagcaaaaagtttcctttttgctgttggagcatgggggttc
agggggtgcagtatctgacgtcaatgccgagcgaaagcgagccgaagg
gtagcatttacgttagataaccccctgatatgaccgacgattatatag
aaaagaagattcaactaggtaaaatcttaatataggttgagatgataa
ggtttataaggaatttgtttgttctaatttttcactcattttgttcta
atttcttttaacaaatgttcttttttttttagaacagttatgatatag
ttagaatagtttaaaataaggagtgagaaaaagatgaaagaaagatat
ggaacagtctataaaggctctcagaggctcatagacgaagaaagtgga
gaagtcatagaggtagacaagttataccgtaaacaaacgtctggtaac
ttcgtaaaggcatatatagtgcaattaataagtatgttagatatgatt
ggcggaaaaaaacttaaaatcgttaactatatcctagataatgtccac
ttaagtaacaatacaatgatagctacaacaagagaaatagcaaaagct
acaggaacaagtctacaaacagtaataacaacacttaaaatcttagaa
gaaggaaatattataaaaagaaaaactggagtattaatgttaaaccct
gaactactaatgagaggcgacgaccaaaaacaaaaatacctatactcg
aatttgggaactttgagcaagaggcaaatgaaatagattgacctccca
ataacaccacgtagttattgggaggtcaatctatgaaatgcgattaag
atagatggctgcaggtcgacggatccccgggaattcactggccgtcgt
tttacaacgtcgtgactgggaaaaccctggcgttacccaacttaatcg
ccttgcagcacatccccctttcgccagctggcgtaatagcgaagaggc
ccgcaccgatcgccctcccaacagttgcgcagcctgaatggcgaatgg
cgcctgatgcggtattttctccttacgcatctgtgcggtatttcacac
cgcatatggtgcactctcagtacaatctgctctgatgccgcatagtta
agccagccccgacacccgccaacacccgctgacgcgccctgacgggct
tgtctgctcccggcatccgcttacagacaagctgtgaccgtctccggg
agctgcatgtgtcagaggttttcaccgtcatcaccgaaacgcgcgaga
cgaaagggcctcgtgatacgcctatttttataggttaatgtcatgata
ataatggtttcttagacgtcaggtggcacttttcggggaaatgtgcgc
ggaacccctatttgtttatttttctaaatacattcaaatatgtatccg
ctcatgagacaataaccctgataaatgcttcaataatattgaaaaagg
aagagtatgagtattcaacatttccgtgtcgccatattccatttttgc
ggcattttgccttcctgtttttgctcacccagaaacgctggtgaaagt
aaaagatgctgaagatcagttgggtgcacgagtgggttacatcgaact
ggatctcaacagcggtaagatccttgagagttttcgccccgaagaacg
ttttccaatgatgagcacttttaaagttctgctatgtggcgcggtatt
atcccgtattgacgccgggcaagagcaactcggtcgccgcatacacta
ttctcagaatgacttggttgagtactcaccagtcacagaaaagcatct
tacggatggcatgacagtaagagaattatgcagtgctgccataaccat
gagtgataacactgcggccaacttacttctgacaacgatcggaggacc
gaaggagctaaccgcttttttgcacaacatgggggatcatgtaactcg
ccttgatcgttgggaaccggagctgaatgaagccataccaaacgacga
gcgtgacaccacgatgcctgtagcaatggcaacaacgttgcgcaaact
attaactggcgaactacttactctagatcccggcaacaattaatagac
tggatggaggcggataaagttgcaggaccacttctgcgctcggccctt
ccggctggctggtttattgctgataaatctggagccggtgagcgtggg
tctcgcggtatcattgcagcactggggccagatggtaagccctcccgt
atcgtagttatctacacgacggggagtcaggcaactatggatgaacga
aatagacagatcgctgagataggtgcctcactgattaagcattggtaa
ctgtcagaccaagtttactcatatatactttagattgatttaaaactt
catttttaatttaaaaggatctaggtgaagatcctttttgataatctc
atgaccaaaatcccttaacgtgagttttcgttccactgagcgtcagac
cccgtagaaaagatcaaaggatcttcttgagatcctttttttctgcgc
gtaatctgctgatgcaaacaaaaaaaccaccgctaccagcggtggttt
gtttgccggatcaagagctaccaactctttttccgaaggtaactggct
tcagcagagcgcagataccaaatactgtccttctagtgtagccgtagt
taggccaccacttcaagaactctgtagcaccgcctacatacctcgctc
tgctaatcctgttaccagtggctgctgccagtggcgataagtcgtgtc
ttaccgggttggactcaagacgatagttaccggataaggcgcagcggt
cgggctgaacggggggttcgtgcacacagcccagcttggagcgaacga
cctacaccgaactgagatacctacagcgtgagctatgagaaagcgcca
cgcttcccgaagggagaaaggcggacaggtatccggtaagcggcaggg
tcggaacaggagagcgcacgagggagatccagggggaaacgcctggta
tctttatagtcctgtcgggtttcgccacctctgacttgagcgtcgatt
tttgtgatgctcgtcaggggggcggagcctatggaaaaacgccagcaa
cgcggcctttttacggttcctggccttttgctggccttttgctcacat
gttctttcctgcgttatcccctgattctgtggataaccgtattaccgc
ctttgagtgagctgataccgctcgccgcagccgaacgccgagcgcagc
gagtcagtgagcgaggaagcggaaga.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A method for producing one or more fermentation end-products by fermenting a lignocellulosic biomass comprising hexose and pentose saccharides with a first microorganism, wherein said first microorganism simultaneously hydrolyses and ferments the lignocellulosic biomass to produce a fermentation end-product.

2. The method of claim 1, wherein at least one of the fermentation end-products is ethanol and wherein the ethanol is produced to a titer of at least about 45 g/L.

3. The method of claim 1, wherein said first microorganism is a Clostridium strain.

4. The method of claim 3, wherein said Clostridium strain is Clostridium phytofermentans.

5. The method of claim 1, wherein the method further comprises the fermentation of hexose and pentose saccharides using a second microorganism.

6. The method of claim 5, wherein the second microorganism is Saccharomyces cerevisiae, C. thermocellum, C. acetobutylicum, C. cellovorans, or Zymomonas mobilis.

7. The method of claim 1, wherein the hexose saccharides comprise carbohydrates selected from the group consisting of cellulose, hemicellulose, starch, mannan, fructose, glucose, galactose, rhamnose, and mannose.

8. The method of claim 1, wherein the pentose saccharides comprise carbohydrates selected from the group consisting of xylan, hemicellulose, xylose, and arabinose.

9. The method of claim 4, wherein the Clostridium phytofermentans is nonrecombinant or recombinant microorganism.

10. The method of claim 4, wherein the Clostridium phytofermentans comprises one or more heterologous polynucleotides.

11. The method of claim 1, further comprising adding one or more medium supplements comprising hexose or pentose saccharides to the medium during the growth of the first microorganism.

12. The method of claim 11, wherein the hexose or pentose saccharides are added in relation to the amount of sugar converted by the first microorganism to other compounds.

13. The method of claim 1, further comprising pretreatment of the biomass.

14. The method of claim 1, wherein said pretreatment comprises steam explosion or hot water extraction, exposure to acid or alkaline conditions.

15. The method of claim 1, further comprising adding a fermentation medium supplement, wherein said fermentation medium supplement is fatty acid, a surfactant, a chelating agent, vitamins, minerals, pH modifiers, yeast extract, and salts.

16. The method of claim 1, wherein said first microorganism simultaneously ferments said hexose and pentose saccharides.

17. The method of claim 1, further comprising adding one or more enzymes, wherein the one or more enzymes are not derived from first microorganism.

18. The method of claim 17, wherein said one or more enzymes is a cellulase, a hemicellulase, a galacturonase, a pectate lyase, a carbohydrase, a xylanase, a glucanase, and endoglucanase, an exoglucanase, a glucosidase, an amylase, a phytase, or a laccase.

19. The method of claim 1, wherein the hexose and pentose saccharides comprise malt syrup, corn steep liquor, distillers dried grains or corn steep solids.

20. The method of claim 1, wherein the method further comprises fed-batch fermentation of biomass with bolus addition of biomass solids.

21. The method of claim 1, wherein said biomass solids are recovered using a sieve.

22. The method of claim 21, wherein the sieve comprises a plurality of apertures between about 150-250 microns in diameter.

23. A biofuel product produced by culturing a strain of Clostridium phytofermentans in a medium comprising a lignocellulosic biomass; wherein the Clostridium phytofermentans simultaneously hydrolyses and ferments the lignocellulosic biomass.

24. A method of producing ethanol, the method comprising the steps of:
a) culturing a strain of Clostridium phytofermentans in a medium comprising a lignocellulosic biomass; wherein the Clostridium phytofermentans simultaneously hydrolyses and ferments the lignocellulosic biomass; and
b) producing ethanol at a yield greater than about 45 g/L.

25. The method of claim 24, further comprising adding one or more medium supplements to the medium during the growth of the Clostridium phytofermentans, wherein one or more of the medium supplements comprises one or more hexose and/or pentose sugar compounds, and the one or more sugar compounds are added in relation to the amount of sugar converted by the Clostridium phytofermentans to other compounds.

26. A system for the production of a fermentation end product comprising:
(a) a fermentation vessel;
(b) a lignocellulosic biomass; and
(c) a first microorganism that simultaneously hydrolyses and ferments the lignocellulosic biomass; wherein the fermentation vessel is adapted to provide suitable conditions for the simultaneous hydrolysis and fermentation of the lignocellulosic biomass.

27. The system of claim 26, further comprising a medium supplement comprising with hexose and pentose saccharides.

28. The system of claim 26, wherein said first microorganism is a Clostridium strain.

29. The system of claim 26, wherein said Clostridium strain is Clostridium phytofermentans.

30. The system of claim 29, wherein the Clostridium phytofermentans comprises one or more heterologous polynucleotides.

31. The system of claim 26, wherein the biomass is pretreated by steam explosion or hot water extraction, exposure to acid or alkaline conditions before contact with the first microorganism.

32. The system of claim 31, wherein the pre-treated biomass is further treated with one or more enzymes not derived from the first microorganism.

33. The system of claim 27, wherein the hexose and pentose saccharides comprise one or more of corn steep solids, corn steep liquor, malt syrup, xylan, cellulose, hemicellulose, fructose, glucose, mannose, rhamnose, or xylose.

34. The system of claim 26, wherein the fermentation medium further comprises one or more enzymes not derived from the first microorganism.

35. The system of claim 26, wherein the fermentation medium further comprises a fermentation medium supplement selected from the group consisting of a fatty acid, a surfactant, a chelating agent, vitamins, minerals, pH modifiers, yeast extract, and salts.

36. The system of claim 26, further comprising a second microorganism.

37. The system of claim 36, wherein the second microorganism is Saccharomyces cerevisiae, C. thermocellum, C. acetobutylicum, C. cellovorans, or Zymomonas mobilis.


SEQUENCE LISTING (1 sequence)

SEQ ID NO: 1 - DNA, 4904 bp, Artificial Sequence
  Description of Artificial Sequence Synthetic polynucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaaagctttggctaacacacacgccattccaaccaatagttttctcggcataaagccatgctctgacgcttaaatgcactaatgccttaaaaaaacattaaagtctaacacactagacttatttacttcgtaattaagtcgttaaaccgtgtgctctacgaccaaaagtataaaacctttaagaactttcttttttcttgtaaaaaaagaaactagataaatctctcatatcttttattcaataatcgcatcagattgcagtataaatttaacgatcactcatcatgttcatatttatcagagctccttatattttatttcgatttatttgttatttatttaacatttttctattgacctcatcttttctatgtgttattcttttgttaattgtttacaaataatctacgatacatagaaggaggaaaaactagtatactagtatgaacgagaaaaatataaaacacagtcaaaactttattacttcaaaacataatatagataaaataatgacaaatataagattaaatgaacatgataatatctttgaaatcggctcaggaaaagggcattttacccttgaattagtacagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatttggtaatataccttataacataagtacggatataatacgcaaaattgtttttgatagtatagctgatgagatttatttaatcgtggaatacgggtttgctaaaagattattaaatacaaaacgctcattggcattatttttaatggcagaagttgatatttctatattaagtatggttccaagagaatattttcatcctaaacctaaagtgaatagctcacttatcagattaaatagaaaaaaatcaagaatatcacacaaagataaacagaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtacctattttttgtgaatcgatccggccagcctcgcagagcaggattcccgttgagcaccgccaggtgcgaataagggacagtgaagaaggaacacccgctcgcgggtgggcctacttcacctatcctgcccggatcgattatgtcttttgcgcattcacttcttttctatataaatatgagcgaagcgaataagcgtcggaaaagcagcaaaaagtttcctttttgctgttggagcatgggggttcagggggtgcagtatctgacgtcaatgccgagcgaaagcgagccgaagggtagcatttacgttagataaccccctgatatgctccgacgctttatatagaaaagaagattcaactaggtaaaatcttaatataggttgagatgataaggtttataaggaatttgtttgttctaatttttcactcattttgttctaatttcttttaacaaatgttcttttttttttagaacagttatgatatagttagaatagtttaaaataaggagtgagaaaaagatgaaagaaagatatggaacagtctataaaggctctcagaggctcatagacgaagaaagtggagaagtcatagaggtagacaagttataccgtaaacaaacgtctggtaacttcgtaaaggcatatatagtgcaattaataagtatgttagatatgattggcggaaaaaaacttaaaatcgttaactatatcctagataatgtccacttaagtaacaatacaatgatagctacaacaagagaaatagcaaaagctacaggaacaagtctacaaacagtaataacaacacttaaaatcttagaagaaggaaatattataaaaagaaaaactggagtattaatgttaaaccctgaactactaatgagaggcgacgaccaaaaacaaaaatacctcttactcgaatttgggaactttgagcaagaggcaaatgaaatagattgacctcccaataacaccacgtagttattgggaggtcaatctatgaaatgcgattaagcttagcttggctgcaggtcgacggatccccgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgccgagcgcagcgagtcagtgagcgaggaagcggaaga